(19) United States
(12) Patent Application Publication
(10) Pub. No.: US 2025/0073283 A1
(43) Pub. Date: Mar. 6, 2025

(54) STREPTOCOCCUS FOR USE IN THE TREATMENT OF GLUCOSE INTOLERANCE

(71) Applicant: INSTITUT NATIONAL DE RECHERCHE POUR L’AGRICULTURE, L'ALIMENTATION ET L'ENVIRONNEMENT, Paris (FR)

(72) Inventors: Christine DELORME, PALAISEAU (FR); Veronique DOUARD, Bures sur Yvette (FR)

(21) Appl. No.: 18/248,147

(22) PCT Filed: Oct. 6, 2021
(86) PCT No.: PCT/EP2021/077574

§ 371 Date: Apr. 6, 2023

(30) Foreign Application Priority Data
Oct. 7, 2020 (FR) FR2010234

(51) Int. Cl. A61K 35/744; A61P 3/10

(52) U.S. Cl. CPC A61K 35/744; A61P 3/10

(57) ABSTRACT

The present invention relates to an inactivated streptococcus, or a membrane fraction derived therefrom, the streptococcus being obtained from a streptococcus comprising a gene encoding a 16S ribosomal RNA, the gene comprising a nucleotide sequence consisting of the sequence SEQ ID NO: 1, for use in a therapeutic method for preventing and/or treating glucose intolerance and/or a disease associated with glucose intolerance in an individual in need thereof.

### TECHNICAL FIELD

The present invention relates to the field of the regulation of glucose homeostasis, in particular regulation of glycemia, and in particular glucose intolerance. More particularly, it describes the use of inactivated bacteria of the genus Streptococcus, or of membrane fractions derived therefrom, the streptococci comprising a gene coding for a 16S ribosomal RNA, said gene comprising a nucleotide sequence consisting of the sequence SEQ ID NO: 1, for improving glucose intolerance, and for preventing and/or treating metabolic diseases associated with glucose intolerance.

### PRIOR ART

Type 2 diabetes (DT2) affects the vast majority of people with diabetes throughout the world (World Health Organization. 1999. Definition, diagnosis and classification of diabetes mellitus and its complications: report of a WHO consultation. Part 1, Diagnosis and classification of diabetes mellitus). Diabetes is a real public health problem owing to its global prevalence, which doubled between 1980 and 2014, increasing from 4.7 to 8.5% of the adult population (World diabetes report, World Health Organization 2016, ISBN: 9789242565256), the high cost of treatment and its complications. In fact, diabetes may in the long term affect the heart, blood vessels, eyes, kidneys and nerves, and may increase the risk of cardiopathy and myocardial infarction. Consequently, prevention and treatment of type 2 diabetes is a highly relevant field of therapeutic research with considerable economic and societal challenges (Diabetes Atlas of the IDF—8th Edition, 2017).

Nowadays, recommendations for measures relating to lifestyle and diet target prediabetes, and although not often prescribed, preventive treatments are recommended for people at high risk of developing type 2 diabetes (Brookshier et al., P-0932 IDF Congress, 2019; Moin et al., Ann Intern Med. 2015). Prediabetes, the early, reversible phase of diabetes, is characterized in particular by glucose intolerance.

Glucose intolerance is a risk factor for type 2 diabetes, which develops in the prediabetes phase. Glucose intolerance or prediabetes or intermediate hyperglycemia is a glycemia disorder. An individual with glucose intolerance has glycemia that is higher than normal but is not yet high enough for a diagnosis of diabetes. It is possible to characterize glucose intolerance by the detection of a decrease in insulin sensitivity or an increase in insulin resistance.

Prediabetes is a phase during which it is often still possible to reverse the diabetic tendency so as to return to a normal glycemia level. This precursor state of type 2 diabetes may be reversible with physical exercise and adjustment of diet.

In general, physical activity combined with a balanced diet may allow blood glucose to be lowered and may prevent progression to type 2 diabetes. However, the estimated increase in cases of type 2 diabetes in the coming years suggests that these adjustments of lifestyle are not sufficient. Thus, it appears that a rebalancing of lifestyle is often unrealistic or insufficient and must be combined with oral medication prescribed for controlling the blood glucose level. However, these medications are few in number and some are not advised for children or adolescents, more and more of whom are developing glucose intolerance. For example, metformin, generally used for treating type 2 diabetes, may be prescribed. However, there are contraindications and unwanted side effects, such as nausea, vomiting, diarrhea, abdominal pains or loss of appetite. These undesirable effects are frequently the reason for stopping treatment (McGovern et al. Obesity and Metabolism, 2017). Long-term treatment with metformin is associated with an increase in the risk of deficiency of vitamin B6 and B12, which may be accompanied by neurologic complications (Porter et al. Endocrinol. Metab. 2019).

Early targeting of the reversible prediabetic state, and in particular glucose intolerance, therefore appears to be essential for preventing its progression to type 2 diabetes.

Solutions using microorganisms have been proposed for preventing the development of glucose intolerance. In particular, it has been described that inactivated commensal strains of Akkermansia muciniphila make it possible to decrease the development of obesity caused by a person's high-fat diet, to reduce the development of metabolic problems caused by weight gain, and improve glucose intolerance (Plovier et al., A purified membrane protein from Akkermansia muciniphila or the pasteurized bacterium improves metabolism in obese and diabetic mice. Nature Medicine, 2017, 23(1): 107-116).

Certain streptococci, more particularly Streptococcus salivarius and Streptococcus vestibularis, are regarded as commensal and are sometimes used as probiotics. For example, the strains of Streptococcus salivarius K12 and M18 are marketed as oral probiotics by the company BLIS PROBIOTICS, for example with the trade name UltraBLIS™ for improving the microbiome and the immune state of the upper respiratory tract and the digestive tract. However, certain strains of Streptococcus salivarius are opportunistic pathogens that cause meningitis or bacteriemia in weakened individuals (Srinivasan et al. 2012, PLoS ONE 7(2): e32169; Wilson et al. 2012, Clinical Medicine & Research, 10(1), 15-25). Moreover, WO 2016/185469 teaches reducing the presence of Streptococcus salivarius in people with glucose intolerance.

There is a need for new compounds or compositions for controlling glucose homeostasis.

There is also a need for new compounds or compositions for treating glucose intolerance.

There is a need for new compounds or compositions whose safety profile may make them suitable for administration to children or adolescents.

There is a need for new treatments for preventing and/or treating glucose intolerance. There is also a need for new treatments for preventing and/or treating diseases associated with or resulting from glucose intolerance, such as, in particular, prediabetes. Thus, there is a need for new compounds or compositions for preventing and/or treating prediabetes.

There is a need for treating and/or preventing diseases associated with glucose intolerance, in particular type 2 diabetes.

The aim of the present invention is to meet some or all of these needs.

### DISCLOSURE OF THE INVENTION

The present invention relates to inactivated bacteria of the genus Streptococcus, or membrane fractions derived therefrom, said streptococci being obtained from streptococci comprising at least one gene coding for a 16S ribosomal RNA, said gene comprising a nucleotide sequence consisting of the sequence SEQ ID NO: 1, for preventing and/or treating glucose intolerance and/or a disease associated with glucose intolerance.

As detailed in the examples given hereunder, it was observed by the inventors that inactivated bacteria of the genus Streptococcus, as defined in the present description, in particular inactivated by heat, are capable of reducing glucose intolerance caused by an obesity-inducing diet in an animal model, or even of restoring normal glycemia and glucose homeostasis.

### SUMMARY OF THE INVENTION

According to one of these first aims, the present invention relates to an inactivated streptococcus, or a membrane fraction derived therefrom, said streptococcus being obtained from a streptococcus comprising at least one gene coding for a 16S ribosomal RNA, said gene comprising a nucleotide sequence consisting of the sequence SEQ ID NO: 1, for use in a therapeutic method for preventing and/or treating glucose intolerance and/or a disease associated with glucose intolerance in an individual in need thereof.

The inventors observed, unexpectedly, that inactivated streptococci, as described here, in particular inactivated by heat, are capable of regulating glucose homeostasis. According to one embodiment, bacteria of the species Streptococcus salivarius are suitable for application for the use that is the subject matter of the present disclosure. Surprisingly, and in contrast to the teaching of WO 2016/185469, administration of inactivated bacteria of the genus Streptococcus, in particular Streptococcus salivarius, in particular thermally inactivated, to animals fed a high-fat diet (obesity-inducing diet) is reflected in a reduction in glucose intolerance induced by this diet. The glycemia of the animals treated in this way approaches that of animals receiving a balanced diet. The reduction in glucose intolerance demonstrates regulation of glucose homeostasis, and makes it possible to prevent, or even treat, a prediabetic state. This effect is not observed with the administration of live Streptococcus salivarius. A treatment comprising administration of bacteria of the genus Streptococcus, in particular Streptococcus salivarius, or a membrane fraction obtained therefrom, may advantageously make it possible to prevent type 2 diabetes.

According to another of these aims, an inactivated streptococcus, or a membrane fraction obtained therefrom, is described, said streptococcus being obtained from a streptococcus comprising a gene coding for a 16S ribosomal RNA, said gene comprising a nucleotide sequence consisting of the sequence SEQ ID NO: 1, for use in a therapeutic method for regulating glucose homeostasis, in particular for regulating glycemia. “Regulating glucose homeostasis” or “regulating glycemia” means, in the sense of the description, inducing in an individual, in response to the administration of an active substance such as described here, a change in glucose homeostasis or glycemia so as to bring them closer to the homeostasis or glycemia that a person skilled in the art would describe as normal or standard for said individual, taking into account their age, sex, diet, physical activity, other associated pathological or physiological state, as well as any other physiological or pathological parameter usually considered in this field.

According to another embodiment, an inactivated streptococcus, or a membrane fraction obtained therefrom, is described here, said streptococcus being obtained from a streptococcus comprising a gene coding for a 16S ribosomal RNA, said gene comprising a nucleotide sequence consisting of the sequence SEQ ID NO: 1, for use in a therapeutic method for improving the response to glucose in an individual with glucose intolerance. “Improving the response to glucose” means, in the sense of the description, inducing in an individual, in response to the administration of an active substance such as described here, a change in the response to glucose so as bring it closer to a response that a person skilled in the art would describe as normal or standard for said individual, taking into account their age, sex, diet, physical activity, other associated pathological or physiological state, as well as any other physiological or pathological parameter usually considered in this field.

According to one embodiment, an inactivated streptococcus, or a membrane fraction obtained therefrom, is also described, said streptococcus being obtained from a streptococcus comprising a gene coding for a 16S ribosomal RNA, said gene comprising a nucleotide sequence consisting of the sequence SEQ ID NO: 1 for use in a therapeutic method for preventing and/or treating prediabetes and/or a disease associated with prediabetes in an individual in need thereof.

According to one embodiment, a disease associated with glucose intolerance may be type 2 diabetes.

According to one embodiment, the present description relates to an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, for use in a therapeutic method for preventing and/or treating type 2 diabetes in an individual in need thereof.

According to another embodiment, an individual in need thereof may be a mammal, and in particular may be a human being.

According to another embodiment, an individual in need thereof may be obese.

According to one embodiment, an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, may be inactivated by a treatment selected from: thermal treatment, ultraviolet irradiation, irradiation with gamma rays, acid treatment, or treatment with hydrogen peroxide. In particular, an inactivated streptococcus may be inactivated by a thermal treatment.

According to one embodiment, a thermal treatment may comprise exposure to a temperature varying from about 50° C. to about 80° C., in particular varying from about 60° C. to about 70° C., and preferably at a temperature of about 65° C.

According to one embodiment, a thermal treatment may comprise exposure to a specific temperature for a period of time ranging from about 15 minutes to about 30 minutes, in particular from about 18 minutes to about 25 minutes, and in particular being about 20 minutes.

According to one embodiment, an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, may be administered by the oral route.

According to one embodiment, an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, may be administered at least once daily, and in particular once daily.

According to a variant embodiment, an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, may be administered for at least two consecutive days out of a period of a week, and in particular for at least five consecutive days out of a period of a week.

According to one embodiment, an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, may be administered for at least one to at least fifteen weeks, in particular for at least two to at least twelve weeks, in particular for at least three to at least ten weeks, or even at least four to at least nine weeks and more particularly for at least two weeks.

According to a variant embodiment, an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, may be administered at a daily dose equivalent to a dose from at least about 1.10⁷ CFU to about at least 1.10¹² CFU, in particular from at least about 1.10⁸ CFU to at least about 1.10¹¹ CFU, and in particular from at least about 1.10⁹ CFU to at least about 1.10¹⁰ CFU.

According to another embodiment of the present description, the inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, may be formulated in a composition comprising at least one pharmaceutically or physiologically acceptable vehicle.

According to one embodiment, a composition may be selected from a powder, granules, a food product, a drink, a pharmaceutical product, a nutraceutical, a food additive, a dietary supplement, a soft capsule, and a hard capsule.

According to another embodiment, a composition as described here may further comprise at least one additional active substance selected from: metabolites, antioxidants, fish oils, DHA, EPA, vitamins, minerals, phytonutrients, a protein, a lipid, probiotics (other than a streptococcus as described here, in particular other than a streptococcus of the species salivarius or of the species vestibularis), and combinations thereof.

According to a variant embodiment, a composition as described here may comprise an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, and at least one physiologically or pharmaceutically acceptable vehicle for use in a therapeutic method for preventing and/or treating glucose intolerance and/or a disease associated with glucose intolerance in an individual in need thereof.

According to one embodiment, a therapeutic method is also described for preventing and/or treating glucose intolerance and/or a disease associated with glucose intolerance in an individual in need thereof, comprising at least one step of administration of an inactivated streptococcus, or of a membrane fraction obtained therefrom, to said individual, said streptococcus being obtained from a streptococcus comprising a gene coding for a 16S ribosomal RNA, said gene comprising a nucleotide sequence consisting of the sequence SEQ ID NO: 1. Said method may further comprise a step of observation of an improvement, in particular of a reduction, in glucose intolerance. Moreover, said method may further comprise a step of observation of an improvement, in particular of a reduction, in the symptoms characterizing the disease associated with glucose intolerance, such as prediabetes, or type 2 diabetes.

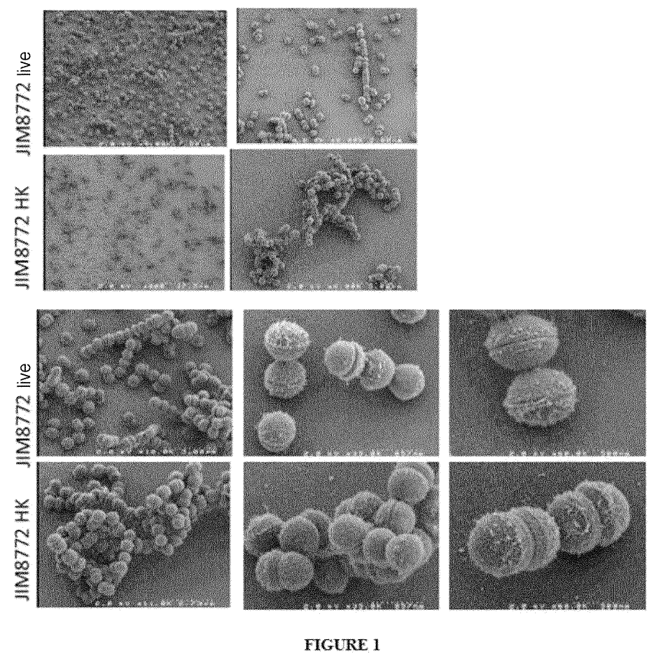

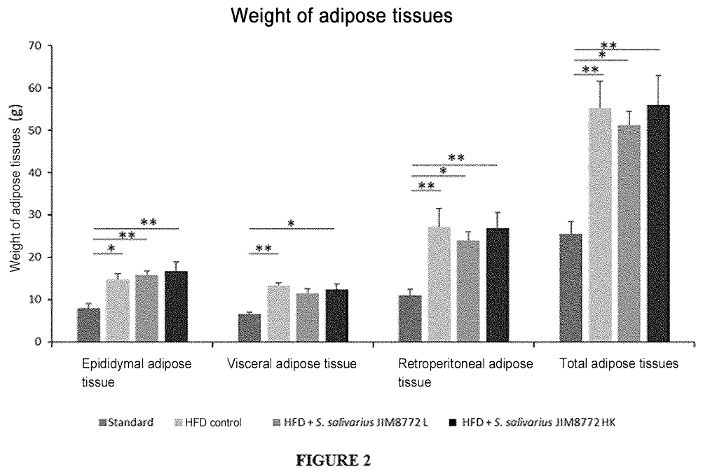

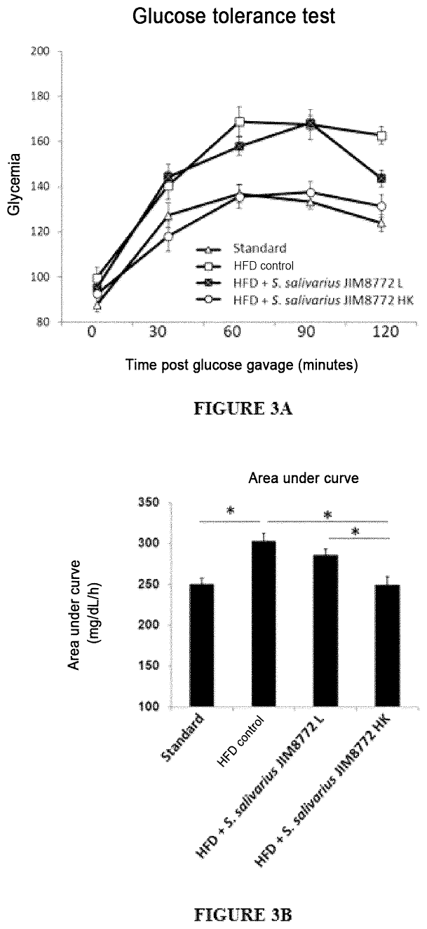

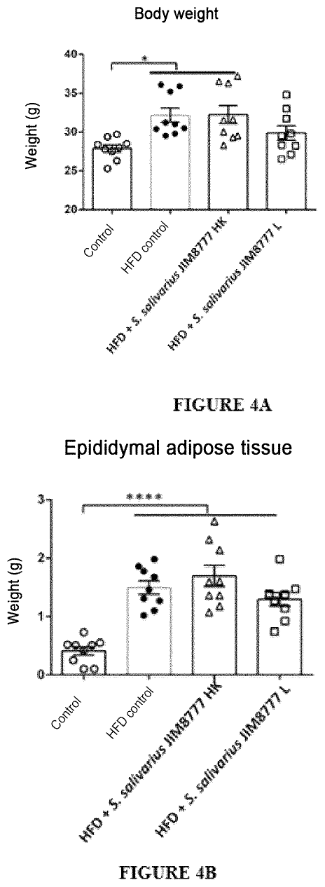

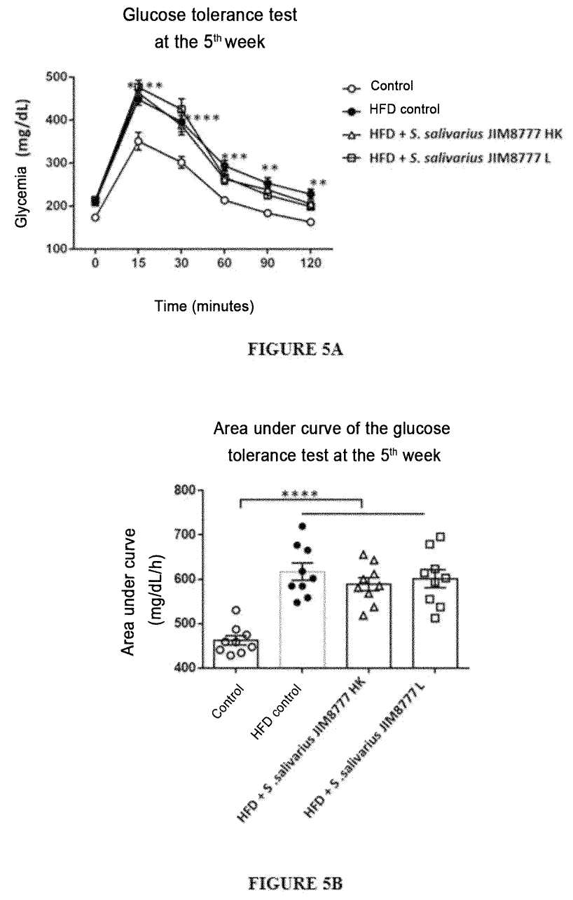

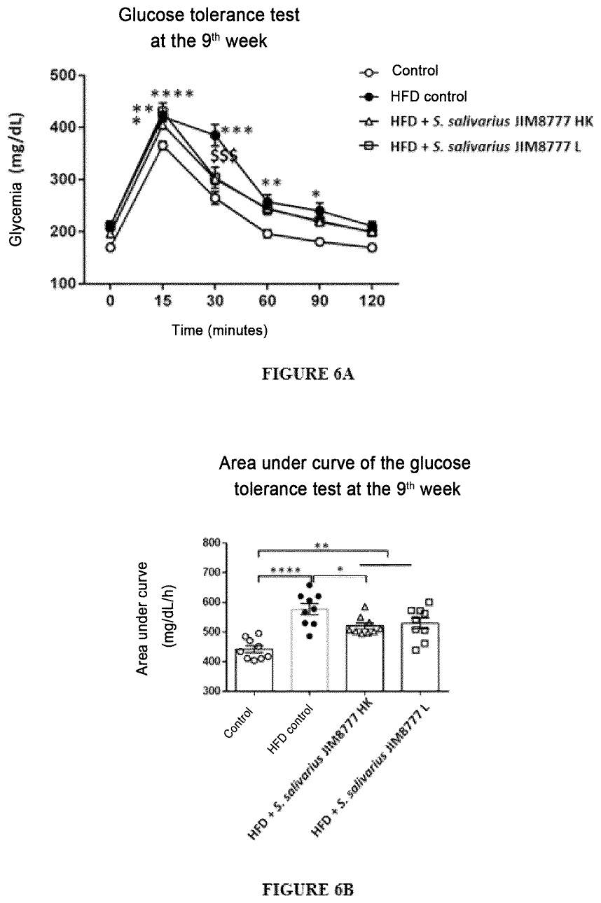

### BRIEF DESCRIPTION OF THE FIGURES

[FIG. 1] shows images in scanning electron microscopy (SEM) of Streptococcus salivarius (strain JIM8772), heat-inactivated (JIM8772 HK) or live (JIM8772 L). Comparison of the images reveals that the thermal inactivation treatment has not altered the morphology and the integrity of the bacteria.

[FIG. 2] shows the final weight of the various adipose tissues (epididymal, visceral and retroperitoneal) and the total adipose mass in rats on a control diet (Standard) or obesity-inducing diet (High Fat Diet (HFD)) for 12 weeks. Ordinate: weight of the adipose tissue (in grams) in rats fed by stomach tube either with a control diet (designated Standard), or a high-fat diet (obesity-inducing diet—HFD), and if applicable supplemented for the last 3 weeks either with a saline solution (HFD Control), or with 1.10¹⁰ CFU of live S. salivarius (HFD+S. salivarius JIM8772 L), or with an equivalent of 1.10¹⁰ CFU of heat-inactivated S. salivarius (HFD+S. salivarius JIM8772 HK). Abscissa: weight of the various adipose tissues and total weight of the sum of these adipose tissues (from left to right): (i) epididymal; (ii) visceral; (iii) retroperitoneal; (iv) total.

[FIG. 3] shows the variation of glycemia over time during an oral glucose tolerance test (OGTT) carried out in the 11th week in rats that had received, for 12 weeks, either a control diet (designated Standard), or a high-fat diet (obesity-inducing diet—HFD), and if applicable supplemented for the last 3 weeks either with a saline solution (HFD Control), or with 1.10¹⁰ CFU of live S. salivarius (HFD+S. salivarius JIM8772 L), or with an equivalent of 1.10¹⁰ CFU of heat-inactivated S. salivarius (HFD+S. salivarius JIM8772 HK). FIG. 3A. Ordinate: glycemia (mg/dL) in rats fed by stomach tube with a diet: (i) Standard (control diet+saline solution−designated Standard)−A); (ii) HFD Control (HFD+saline solution−□); (iii) HFD+live S. salivarius JIM8772 (HFD+S. salivarius JIM8772 L−▪) or (iv) HFD+heat-inactivated S. salivarius JIM8772 (HFD+S. salivarius JIM8772 HK−∘). Abscissa: time in minutes after administration of oral glucose (2 g/kg). FIG. 3B. Ordinate: Area under curve (mg·dl⁻¹·h⁻¹). Abscissa (from left to right): (i) Standard (control diet+saline solution−Standard); (ii) HFD Control (HFD+saline solution); (iii) HFD+live S. salivarius JIM8772 (HFD+S. salivarius JIM8772 L) or (iv) HFD+heat-inactivated S. salivarius JIM8772 (HFD+S. salivarius JIM8772 HK) *p<0.05.

[FIG. 4] shows the total weight (FIG. 4A) and the adipose mass of the epididymis (FIG. 4B) in mice on a normal diet (Control) or obesity-inducing (High Fat Diet (HFD)) for 12 weeks, and if applicable supplemented either with a saline solution (HFD Control), or with 1.10⁹ CFU of live S. salivarius (HFD+S. salivarius JIM8777 L), or with an equivalent of 1.10⁹ CFU of heat-inactivated S. salivarius (HFD+S. salivarius JIM8777 HK). FIG. 4A. Ordinate: body weight (in grams) of the mice. Abscissa: Diets administered to the mice (from left to right): (i) Control (control diet+saline solution−chow−∘); (ii) HFD (HFD+saline solution−●); (iii) HFD+heat-inactivated S. salivarius JIM8777 (HFD+S. salivarius JIM8777 HK Δ); (iv) HFD+live S. salivarius JIM8777 (HFD+S. salivarius JIM8777 L−□)*p<0.05. FIG. 4B. Ordinate: weight (in grams) of epididymal adipose tissue of the mice. Abscissa: Diets administered to the mice (from left to right): (i) Control (control diet+saline solution−∘); (ii) HFD Control (HFD+saline solution−●); (iii) HFD+heat-inactivated S. salivarius JIM8777 (HFD+S. salivarius JIM8777 HK−Δ); (iv) HFD+live S. salivarius JIM8777 (HFD+S. salivarius JIM8777 L−□). ****p<0.0001. [FIG. 5] shows the variation of glycemia over time during an oral glucose tolerance test (OGTT) in mice that had received, for 5 weeks, either a Control diet, or a high-fat diet (obesity-inducing diet−HFD), and if applicable supplemented either with a saline solution (HFD Control), or with an equivalent of 1.10⁹ CFU of heat-inactivated S. salivarius (HFD+S. salivarius JIM8777 HK), or 1.10⁹ CFU of live S. salivarius (HFD+S. salivarius JIM8777L). FIG. 5A. Ordinate: Glycemia (mg/dL) in mice fed by stomach tube with a diet: (i) Control (control diet+saline solution−∘); (ii) HFD Control (HFD+saline solution−●); (iii) HFD+heat-inactivated S. salivarius JIM8777 (HFD+S. salivarius JIM8777 HK−Δ); (iv) HFD+live S. salivarius JIM8777 (HFD+S. salivarius JIM8777 L−□). Abscissa: time in minutes after administration of oral glucose (2 g/kg). **p<0.01; ***p<0.001 and **** p<0.0001 (HFD control/IFD+JIM8777 HK/HFD+JIM8777 L vs Control). FIG. 5B. Ordinate: Area under curve (mg·dl⁻¹·h⁻¹). Abscissa (from left to right): (i) Control (control diet+saline solution−∘); (ii) HFD control (HFD+saline solution−●); (iii) HFD+heat-inactivated S. salivarius JIM8777 (HFD+S. salivarius JIM8777 HK−Δ); (iv) HFD+live S. salivarius JIM8777 (HFD+S. salivarius JIM8777 L−□). ****p<0.0001.

[FIG. 6] shows the variation of glycemia over time during an oral glucose tolerance test (OGTT) in mice that had received, for 9 weeks, either a Control diet, or a high-fat diet (obesity-inducing diet—HFD), and if applicable supplemented either with a saline solution (HFD Control), or with an equivalent of 1.10⁹ CFU of heat-inactivated S. salivarius (HFD+S. salivarius JIM8777 HK), or 1.10⁹ CFU of live S. salivarius (HFD+S. salivarius JIM8777 L). FIG. 6A. Ordinate: Glycemia (mg/dL) in mice fed by stomach tube with a diet: (i) Control (control diet+saline solution−∘); (ii) HFD Control (HFD+saline solution−●); (iii) HFD+heat-inactivated S. salivarius JIM8777 (HFD+S. salivarius JIM8777 HK−Δ); (iv) HFD+live S. salivarius JIM8777 (HFD+S. salivarius JIM8777 L−□). Abscissa: time in minutes after administration of oral glucose (2 g/kg). *p<0.01, **p<0.01; ***p<0.001 and ****p<0.0001 (HFD control/IFD+JIM8777 HK/IFD+JIM8777 L vs Control), $$$ p<0.001 (HFD+JIM8777 HK and HFD+JIM8777 L vs HFD control). FIG. 6B. Ordinate: Area under curve (mg·dl⁻¹·h⁻¹). Abscissa (from left to right): (i) Control (control diet+saline solution−∘); (ii) HFD control (HFD+saline solution−●); (iii) HFD+heat-inactivated S. salivarius JIM8777 (HFD+S. salivarius JIM8777 HK−Δ); (iv) HFD+live S. salivarius JIM8777 (HFD+S. salivarius JIM8777 L−□). *p<0.05; **p<0.01; ****p<0.0001.

### DETAILED DESCRIPTION

As detailed in the examples given hereunder, the inventors observed that, in contrast to live streptococcus, and unexpectedly, a streptococcus comprising a gene coding for a 16S ribosomal RNA, whose sequence comprises a sequence consisting of the sequence SEQ ID NO: 1, and inactivated, in particular inactivated by heat, made it possible to improve glucose intolerance substantially. Thus, an inactivated streptococcus, or a membrane fraction obtained therefrom, said streptococcus being obtained from a streptococcus comprising a gene coding for a 16S ribosomal RNA, said gene comprising a sequence consisting of the sequence SEQ ID NO: 1, may advantageously be used for treating and/or preventing glucose intolerance and/or a disease associated with glucose intolerance. More particularly, the inventors have demonstrated the capacity of various inactivated strains of S. salivarius, JIM8772 and JIM8777, to improve or prevent glucose intolerance in two animal models, rat and mouse, fed a high-fat diet (obesity-inducing diet).

Advantageously, an inactivated streptococcus, or a membrane fraction obtained therefrom, said streptococcus being obtained from a streptococcus comprising a gene coding for a 16S ribosomal RNA, said gene comprising a sequence consisting of the sequence SEQ ID NO: 1, may be used for regulating the glycemic response of an individual. Also, advantageously, an inactivated streptococcus, or a membrane fraction obtained therefrom, said streptococcus being obtained from a streptococcus comprising a gene coding for a 16S ribosomal RNA, said gene comprising a sequence consisting of the sequence SEQ ID NO: 1, may be used for normalizing the glycemia of an individual with glucose intolerance.

### Definitions

The terms used in the present description are used with their usual meaning in the technical field considered, and having regard to the context of the description in which the terms are used. Certain terms are discussed in more detail hereunder, or elsewhere in the description, to supply additional information regarding the present description and its implementation. The following definitions are supplied for the description and the claims.

The description of the various embodiments of the present description comprises the embodiments including “comprising”, “having”, “consisting of” and “essentially consisting of”. The words “have” and “comprise”, or variants such as “has”, “having”, “comprises” or “comprising” are to be understood as implying the inclusion of the element or elements indicated (such as an element of a composition or a step of a method) but not the exclusion of other elements. The term “consisting of” implies inclusion of the element or elements indicated, excluding any additional element. The expression “essentially consisting of” implies inclusion of the elements indicated, and optionally other elements when the other elements do not materially affect the fundamental, new features as described here. Depending on the context, the term “comprise” may also strictly indicate the features indicated, the integers, the steps or the components indicated and, consequently, in this case it may be replaced with “consist of”.

“Type 2 diabetes” or “DT2” denotes a chronic disease that develops when the pancreas does not produce sufficient insulin (glycemia regulatory hormone), or when the body is unable to make effective use of the insulin that it produces. DT2 is generally preceded by glucose intolerance.

The terms “about” or “approximately” as used here with respect to a numerical value refer to the usual error range for the value in question, as usually identified by a person skilled in the art in the technical field considered. The mention of the term “about” with respect to a value or a specific parameter includes and describes this value or this parameter as such. The term “about” refers to ±10% of a given value. However, whenever the value in question refers to an indivisible object that would lose its identity once subdivided, “about” then refers to ±1 of the indivisible object.

The term “individual” or “patient” as used in the present text denotes a mammal in particular. The mammals considered include, but are not limited to, domestic animals (for example cattle, sheep, cats, dogs, and horses), primates (for example human and nonhuman), rabbits and rodents (for example, mice and rats). According to a particular embodiment, an individual, or patient, is a human being.

“Glucose intolerance” means a pathophysiological state in which an individual's glycemia is above normal but below the threshold for a diagnosis of type 2 diabetes. Glucose intolerance may be identified by, among others, an oral glucose tolerance test (OGTT). “Obesity” means a pathophysiological state in which an individual has, in particular, an excess of adipose tissue, generally induced by a diet described as obesity-inducing, comprising, in particular, an excessive consumption of high-calorie foods, genetic predisposition, or insufficient or nonexistent sporting activity. An individual stated to be obese has a body mass index (BMI) above 30. Body mass index, according to an official definition of the World Health Organization (WHO), is the indicator of the health risks associated with being overweight or underweight. The BMI is calculated by dividing the individual's weight (in kilograms) by their height (in meters) squared. A BMI value is associated with a specific build according to the classification given by the WHO.

“Prediabetes” means a pathophysiological state characterized in particular by a high glycemia value compared to normal, but below the threshold for defining type 2 diabetes. Fasting glycemia is considered to be (i) normal between 0.70 and 1.10 g/l, (ii) a sign of prediabetes between 1.11 and 1.25 g/l and (iii) a sign of diabetes when >1.25 g/l. Prediabetes does not generally cause symptoms, but is often associated with obesity, dyslipidemia, and hypertension. It is a risk factor for cardiovascular diseases. Prediabetes is characterized in particular by glucose intolerance.

In the context of the present invention, the terms “prevent”, “prevention” and “slow the progression of” (and variants of these expressions) with regard to a physiological disorder or a disease relates to the prophylactic treatment of the disease or disorder, for example in an individual suspected of having this disease or this disorder, or being at risk of developing this disease or this disorder. “Prevent” includes, but is not limited to, the prevention or slowing of the development of the disease, and/or keeping one or more disease symptoms at a desired level or a lower level. The term “prevent” does not require 100% elimination of the possibility or probability of occurrence of the disease or disorder. This term rather denotes reduction, to a lesser degree, of the risk or probability of occurrence of a given phenomenon, that is, in the present invention, of glucose intolerance or of a disease associated with glucose intolerance, such as a prediabetic state or type 2 diabetes. As indicated, prevention may be complete, i.e. absence of symptoms or of detectable disease, or partial, so that there are fewer symptoms or the symptoms are of lower intensity.

“Glucose homeostasis” means an individual's glycemic balance, characteristic of normal glycemia. The normal fasting glycemic balance in humans is between about 0.70 g/L and about 1.10 g/L (i.e. between 70 mg/dL and 110 mg/dL). Glycemia below 0.70 g/L characterizes hypoglycemia, whereas glycemia above 1.10 g/L but below 1.26 g/L characterizes moderate hyperglycemia, which may reveal glucose intolerance.

As used here, the terms “therapeutically effective amount” and “prophylactically effective amount” relate to an amount that provides a therapeutic advantage in the treatment, prevention or management of the pathologic processes considered. The specific amount that is therapeutically effective can easily be determined by a physician and may vary as a function of factors such as the type and the stage of the pathologic processes considered, the patient's medical history, sex, weight and age, their diet, and the administration of other therapeutic agents.

In the sense of the present description, “significantly” used in the context of a change, signifies that the change observed is noteworthy or that it is statistically significant.

In the sense of the present description the expressions “substantially similar”, “not substantially different”, “roughly similar” or “not substantially different” (or any other variant) used in connection with a feature as described here aim to a define a set of embodiments in connection with this feature which are broadly but not completely identical to the embodiments that include this feature.

“Oral glucose tolerance test” or “OGTT” means a test for measuring the body's ability to use glucose. An OGTT is familiar to a person skilled in the art. It is possible for example to use the protocol described in particular in Nagy et al., 2018.

According to the present description, the terms “treat”, “treatment”, “therapy” or “therapeutic” relate to the administration or ingestion of an active substance as described herein, or of a composition comprising said active substance, for purposes of cure, relief, reduction, attenuation, or improvement of a disease or of a pathologic disorder, or of one or more associated symptoms, or for preventing or slowing the progression of this or these symptoms or of this disease, or for stopping the development of this or these symptoms, or of this disease or of this pathologic disorder in a statistically significant manner. More particularly, “treat” or “treatment” include any approach for obtaining a beneficial effect or a desired result with respect to a state of glucose intolerance in an individual. The beneficial or desired clinical results may include, but are not limited to, attenuation or improvement of glucose intolerance, of a disease associated with glucose intolerance, such as prediabetes or type 2 diabetes, or of one or more symptoms of such a disease; decrease or reduction of the extent of glucose intolerance, of a disease associated with glucose intolerance, or of one or more symptoms of such a disease; stabilization, i.e. absence of aggravation, of glucose intolerance, of a disease associated with glucose intolerance, or of one or more symptoms of such a disease; prevention of glucose intolerance, of a disease associated with glucose intolerance, or of one or more symptoms of such a disease; prevention of the spreading of a disease associated with glucose intolerance, or of one or more symptoms of such a disease; slowing of a disease associated with glucose intolerance, or of one or more symptoms of such a disease or progression of one or more symptoms of such a disease; decrease of the reappearance of glucose intolerance, of a disease associated with glucose intolerance, or of one or more symptoms of such a disease; and the interruption of glucose intolerance, of a disease associated with glucose intolerance, or of one or more symptoms of such a disease. In other words, “treatment” as used here comprises any cure, improvement, reduction or interruption of glucose intolerance, of a disease associated with glucose intolerance, or of one or more symptoms of such a disease. A “reduction” of a symptom or of a disease signifies a decrease in the severity or frequency of the disease or symptom, or elimination of the disease or symptom.

As used in this description and the claims, the singular forms “a” and “the” include the plural, unless explicitly indicated otherwise by the context.

The expression “physiologically acceptable vehicle” denotes any substance or composition compatible with the organism of the individual that is to be administered the active substance presented in the present description. In particular, a physiologically acceptable vehicle is a substance or composition whose administration to an individual is not accompanied by significant harmful effects. It may, for example, be a nontoxic solvent such as water or an aqueous saline solution. In particular, said vehicle is compatible with oral or rectal administration, and preferably is suitable for administration by the oral route.

The sources, ingredients and components listed hereunder are understood as being described in such a way that all combinations and mixtures thereof are also envisaged in the context of the present invention.

It is to be understood that every maximum numerical limitation given in the description comprises every lower numerical limitation, as if these lower numerical limitations were written expressly. Every minimum numerical limitation given in this description comprises any upper numerical limitation, as if these upper numerical limitations were written expressly here. Every numerical range given throughout the description comprises every narrower numerical range included in said wider numerical range, as if these narrower numerical ranges were all written expressly.

All the lists stated in the description, such as for example the lists of ingredients, are intended and are to be interpreted as Markush groups. Thus, all the lists may be read and interpreted as elements “selected from the group consisting of” . . . list of the elements . . . “and combinations and mixtures thereof”.

Reference may be made hereunder to trade names of components comprising various ingredients used in the present description. The inventors do not intend to be limited to materials under a particular trade name. Equivalent materials (for example, those obtained from a different source under a different name or reference number) to those indicated here by a trade name may be substituted and used in the description hereunder.

As used in the present description, the term “gene coding for a 16S rRNA” or “gene coding for a 16S ribosomal RNA” consists of a nucleic acid of the bacterial genomic DNA, whose nucleic acid sequence is completely complementary to the natural 16S rRNA. A gene coding for a 16S rRNA may be sequenced using the primers V3F ((5′-TACGGRAGGCAGCAG-3′-SEQ ID NO: 4) and V4R (5′-TACCAGGGTATCTAATCCT-3′-SEQ ID NO: 5) (Kozich et al. (2013) Appl. Environ. Microbiol. 79, 5112-5120) and Takara Ex Taq polymerase (Takara) for amplification of the DNA of a pure colony. Alternatively, a gene coding for a 16S rRNA comprising a sequence consisting of the nucleotide sequence of SEQ ID NO: 1, or comprising one of the sequences SEQ ID NO: 2 or 3, may be amplified and then sequenced using the primers SEQ ID NO: 6 (5′-gctaataccgcataacaatgg-3′) and SEQ ID NO: 7 (5′-cggaaaggatccaacaccta-3′). The terms “16S rRNA” or “16S ribosomal RNA” are used in the present description according to their meaning commonly assumed in this field and aim to denote the ribosomal RNA of the 30S subunit of a prokaryotic ribosome.

Inactivated streptococcus

### Bacteria of the Genus Streptococcus

The streptococci according to the present description are streptococci, or bacteria of the genus Streptococcus, comprising at least one gene coding for a 16S ribosomal RNA whose sequence comprises a sequence consisting of the sequence SEQ ID NO: 1.

The streptococci may be bacteria belonging to the species S. salivarius, S. vestibularis, or to a mixture thereof.

According to one embodiment, the streptococci may be bacteria of the species Streptococcus salivarius.

According to one embodiment, the streptococci may comprise a gene coding for a 16S ribosomal RNA whose sequence comprises a sequence consisting of the sequence SEQ ID NO: 2.

According to one embodiment, the streptococci may comprise a gene coding for a 16S ribosomal RNA whose sequence comprises a sequence consisting of the sequence SEQ ID NO: 3.

The bacteria suitable for application according to the description may be bacteria that are or are not genetically modified. The preparation of said bacteria forms part of the general knowledge in this field. It is thus possible to refer to the manual: “The GMO Handbook: Genetically Modified Animals, Microbes, and Plants in Biotechnology” (Humana Press 2004; DOI https://doi.org/10.1007/978-1-59259-801-4), which describes various methods of producing genetically modified microorganisms.

The gene coding for the 16S rRNA used for defining the streptococci suitable for the present description is not genetically modified. “Not genetically modified” with regard to a given nucleotide sequence means a sequence that exists naturally in the bacterium in question (which has not been introduced artificially), in its genome, chromosome or plasmid, and that may be used for genetically characterizing or identifying this bacterium. “Not genetically modified” with regard to a given bacterium means a bacterium that exists naturally and whose genome, chromosome or plasmid has not been modified artificially by genetic engineering. “Genetic engineering” denotes all of the tools and technical methods for modifying the genetic constitution of an organism by deletion, addition or substitution of one or more (at least one) nucleotide of its genome.

A streptococcus suitable for application of the invention described here is a streptococcus comprising a gene coding for a 16S ribosomal RNA whose sequence comprises a nucleotide sequence consisting of the sequence SEQ ID NO: 1, the gene not being modified by genetic engineering. Said sequence therefore comprises neither addition, nor deletion, nor substitution of nucleotide(s), carried out by genetic engineering, compared to the nucleotide sequence that exists naturally in the species or genus of streptococcus in question, and has not been introduced artificially into the bacterium. Said streptococcus may further comprise modifications (addition, deletion, substitution), carried out by genetic engineering, in one or more nucleotide sequences of its genome, other than the sequence or sequences of the gene coding for 16S rRNA. A streptococcus suitable for the invention is a streptococcus whose gene or genes coding for 16S rRNA exist(s) naturally in this streptococcus. Said gene has therefore not been introduced into the bacterium artificially.

According to a variant embodiment, a streptococcus suitable for application of the invention described here is a streptococcus comprising one or more gene(s) coding for a 16S ribosomal RNA whose sequence comprises a nucleotide sequence consisting of the sequence SEQ ID NO: 2 or the sequence SEQ ID NO: 3, the gene or genes not being modified by genetic engineering. The gene or the genes have not been introduced artificially in the streptococcus or streptococci in question.

According to a variant embodiment, a streptococcus suitable for application of the invention described here is a streptococcus that has not been modified by genetic engineering. No element of the genome of said streptococcus comprises addition, deletion, or substitution of nucleotide(s) carried out by genetic engineering compared to the nucleic acid sequences existing naturally in the streptococcus species or genus in question.

The gene coding for 16S ribosomal RNA (rRNA) is a gene usually employed as a molecular marker in microbial biology (Giovannoni et al. Nature. 1990; 345(6270): 60-63). The techniques for sequencing the bacterial genes coding for 16S ribosomal RNA are familiar to a person skilled in the art.

As an example, it is possible to use the polymerase chain reaction (PCR), which consists of amplifying a nucleotide sequence of a whole gene or of a part of a gene, with specific primers. As an example, a nucleotide sequence of a gene coding for a 16S rRNA may be amplified using the primers SEQ ID NO: 4 and SEQ ID NO: 5 and a DNA polymerase enzyme, for example Takara Ex Taq polymerase (Takara Bio Inc.), using a thermocycler.

The conditions used may be 95° C. for 15 minutes, followed by 38 cycles of 95° C. for 30 seconds, hybridization at 52° C. for 30 seconds, and extension at 72° C. for 1.5 minutes, with a final extension at 72° C. for 10 minutes for bacterial amplification.

The nucleotide sequence thus amplified is then sequenced (for example by Sanger's method (1976)) and compared against one or more other nucleotide sequences, optionally deposited in data banks (for example, EMBL, NCBI, BiBi, Genebank).

Comparison of a nucleotide sequence (compared sequence) of a gene or of a part of a gene coding for a 16S rRNA against other nucleotide sequences (reference sequence(s)) of a gene or of a part of a gene coding for a 16S rRNA is a method for identifying the bacterium or the bacteria from which the compared nucleotide sequence was derived.

As an example, a nucleotide sequence of a gene coding for a 16S rRNA may be amplified using primers and a DNA polymerase enzyme, using a thermocycler. Beforehand, in the amplification step, the nucleotide sequence to be amplified is isolated from the bacterium, by any means known in this field, for example by extraction of the genome of the bacterium. After the amplification step, the sequence amplified is purified, and then sequenced, for example using a sequencer.

A streptococcus suitable for the invention may be S. salivarius or S. vestibularis. A mixture of S. salivarius and S. vestibularis may also be suitable application of the invention described here.

According to one embodiment, a streptococcus strain may be used in an isolated or purified form, i.e. not mixed with one or more compounds that may be associated with it in its original environment.

The methods of isolation and purification of bacterial strains are familiar to a person skilled in the art and are described in particular in Smith et al. 1993 (Oralstreptococcal colonization ofinfants. Oral. Microbiol. Immunol. 8:1-4).

Isolation of a streptococcus strain may be carried out starting from bacterial cultures or biological samples taken from an individual, in particular a human being, and in particular may be a sample of saliva or of feces.

Strains of S. salivarius

Streptococcus salivarius is a species of bacterium of the genus Streptococcus, belonging to the family Streptococcaceae and to the order Lactobacillales. Streptococcus salivarius is a commensal species in humans. It mainly occurs in the human oral cavity, in the saliva and in the multi-species biofilm on the superior surface of the tongue (Aas et al., 2005, Defining the normal bacterial flora of the oral cavity. Journal of Clinical Microbiology, 43(11), 5721-32), but also in the digestive system of neonates (Carlsson et al. 1970, Early establishment of Streptococcus salivarius in the mouth of infants. Journal of Dental Research, 49(2), 415-8. R), and in human feces.

Any strain or bacterium of the species S. salivarius may be suitable for the invention as described here.

“Bacterium belonging to the species Streptococcus salivarius” means a bacterium of spherical or ovoid form, a facultative anaerobe, of the Gram-positive type, metabolizing lactose and glucose and comprising at least one gene coding for a 16S ribosomal RNA, said gene comprising a nucleotide sequence consisting of the sequence SEQ ID NO: 1.

A strain of S. salivarius that may be suitable for the invention is easily identifiable in the microbiological databases known by a person skilled in the art. In particular, a person skilled in the art may obtain a strain of this kind from the National Center for Biotechnology Information, Collection Institut Pasteur (CIP), from the Belgium Coordinated Collections of Microorganisms (BCCM-LMG), from Paris Cochin Hospital (CCH) or from the National Collection of Type Cultures.

As an example, a person skilled in the art may obtain the strains NCTC 8618 or NCTC 7366, from the National Collection of Type Cultures.

According to one embodiment, a strain of the species S. salivarius may comprise at least one gene coding for a 16S ribosomal RNA and comprising a nucleotide sequence consisting of the sequence SEQ ID NO: 2.

In a nonlimiting manner, a strain of the species S. salivarius that may be suitable for the invention may be selected from: NCTC 8618, FDAARGOS_259, ICDC2, LAB813, ATCC 25975, ICDCI, ICDC3, JF, NCTC7366, ATCC 27945, HSISS4, ATCC 703, 34-09 S8, 20-12 S2, 85-06 524, F6-1, 39-01 S14, BIOML-A22, BIOML-A24, BIOML-A25, BIOML-A19, BIOML-A21, UMB0028, BIOML-A13, BIOML-A16, BIOML-A15, BIOML-A29, BIOML-A36, BIOML-A23, BIOML-A5, BIOML-A3, BIOML-A10, BIOML-A7, UBSSO1, BIOML-A35, KB005, DE0578, BIOML-A4, bf_0095, HS0302, BIOML-A34, BIOML-A28, BIOML-A18, BIOML-A30, AF24-6AC, BIOML-A1, BIOML-A27, BIOML-A26, BIOML-A14, BIOML-A17, UMB0051, BIOML-A32, BIOML-A31, MIT 14-1770-C1, 85-04 S22, MGYG-HGUT-00113, 2202 S3, BIOML-A20, BIOML-A2, YU10, NU10, VA08-2AN, UC3162, AF13-49B, AF23-9AC, 40-02 S18, 1001175st1_H3, AF29-16, BIOML-A33, SS_Bg39, AF10-23, BIOML-A6, BIOML-A9, BIOML-A8, BIOML-A12, 34-24 S10, BCC42, 1003 SOLI, 37-09 S13, AS012762, 85-05 S23, 1270 SSAL, 726 SSAL, GED7778A, 140_SSAL, 85-02 s21, 22-08 S7, 37-08 512, 20-02 Si, 84-12 S20, 39-07 515, 34-19 S9, 39-09 516, UBA10771, 918_SSAL, 57.1, CCHSS3, JIM 8777, JIM 8772, K12, M18, PS4, SK126, and a mixture thereof.

According to one embodiment, a strain of the species S. salivarius may be selected from JIM 8772, JIM 8777, or a mixture thereof.

The strain JIM 8772 is described in Delorme et al. 2007, Extent ofHorizontal Gene Transfer in Evolution of Streptococci of the Salivarius Group. Journal of Bacteriology, February 2007, p. 1330-1341). The strain JIM 8777 is identified by the NCBI number: txid347253. The complete sequence of the genome of this strain is deposited with DDBJ/EMBL/GenBank under accession number FR873482 (Guedon et al. 2011, Complete Genome Sequence of the Commensal Streptococcus salivarius Strain JIM8777. Journal of Bacteriology, Vol. 193, No. 18, p. 5024-5025).

Strains of S. vestibularis

Streptococcus vestibularis is a species of bacterium of the genus Streptococcus, belonging to the family Streptococcaceae and the order Lactobacillales. Streptococcus vestibularis is a commensal species in humans. It mainly occurs in the saliva in the human oral cavity, and in human feces.

Any strain or bacterium of the species S. vestibularis may be suitable for the invention described here.

“Bacterium belonging to the species Streptococcus vestibularis” means a bacterium of spherical or ovoid form, a facultative anaerobe, of the Gram-positive type and comprising at least one gene coding for a 16S ribosomal RNA, said gene comprising a nucleotide sequence consisting of the sequence SEQ ID NO: 1.

A strain of S. vestibularis that may be suitable for the invention is easily identifiable in the microbiological databases known by a person skilled in the art. In particular, a person skilled in the art may obtain a strain of this kind from the National Center for Biotechnology Information, Collection Institut Pasteur (CIP), from the Belgium Coordinated Collections of Microorganisms (BCCM-LMG), from Paris Hospital Cochin (CCH) or from the National Collection of Type Cultures.

More particularly, a bacterium belonging to the species S. vestibularis according to the present description may comprise at least one gene coding for a 16S ribosomal RNA, said gene comprising a nucleotide sequence consisting of the sequence SEQ ID NO: 3.

In a nonlimiting manner, a strain of the species S. vestibularis that may be suitable for the present description may be selected from: NCTC12167, NCTC13732, NCTC1267, ATCC49124, AS012761, OM08-1, MGYG-HGUT-02302, 1005_STHE, 22-04 S5, 22-06 S6, 22-03 S4, ATCC 49124 and F0396 and a mixture thereof.

As an example, a person skilled in the art may obtain bacterial strains according to the present description, such as the strains NCTC12167 or NCTC13732, from the National Collection of Type Cultures.

### Culture of Streptococci

The streptococci used according to the present description may be cultured and amplified by any method known in this field. They may be cultured on a solid culture medium, for example on agar, or in a liquid culture medium. Nonexhaustively, various media suitable for culture of streptococci may be used. As an example, we may mention rich media (Mitis-salivarius agar—Smith et al. 1993 Oral. Microbiol. Immunol; Colombia blood agar, M17, Todd-Hewitt broth—Wescombe et al. 2011. Microbiology), media chemically defined (MCD—Kaci et al. 2011. AEM) or artificial media (artificial saliva—Roger et al. 2011. J. App. Microbiol). These media may be used in a chemostat (Harty et al. 1988. J. App. Microbiol; Chen and Burne, 1995. FEMS Microbiol). The streptococci cultured in a liquid culture medium may be cultured in fermenters, the volumes of which may range from some tens of milliliters to some tens of liters. The operating conditions of said fermenters, temperature, stirring, pH, 02 pressure, CO₂ pressure are known in this field. The end of the culture step may be determined by any method known in this field, for example by measuring the pH of the culture (for example, acidification of the culture medium may indicate that the bacterial biomass has consumed all the available nutrients) or by measuring the optical density.

After the culture step, the streptococci may be inactivated without prior preparation, in the culture medium. Alternatively, the streptococci may be purified before being inactivated. Any method of purification of bacteria known in this field may be used. For example, the culture medium containing the bacteria may be centrifuged, the supernatant removed, and the bacterial pellet resuspended in a buffer, for example NaCl buffer. The bacteria suspended in the buffer may be submitted to a new cycle of centrifugation—resuspension in a buffer. Said cycle may be carried out at least twice, or even three or four times. The volume of buffer used for suspending the bacteria may be adapted so as to dilute or concentrate the bacteria. According to one embodiment, the volume of buffer is adapted to concentrate the bacteria. Other methods of purification by filtration, recovery and washing of the retentate or by dialysis may also be used.

### Inactivation of the Streptococci

The streptococci may be inactivated by any method used in this field. According to one embodiment, the streptococci are inactivated thermally.

In the sense of the present description, inactivated bacteria are bacteria that have been made incapable of growing and dividing. Preferably, inactivated bacteria no longer have metabolic activity. Advantageously, inactivated bacteria according to the present description retain their integrity at the end of the inactivation treatment, and may remain in intact form. In particular, after inactivation the bacteria still have their surface compounds unchanged, or substantially unchanged.

Any methods, treatments, or processes of inactivation of bacteria known by a person skilled in the art may be used for obtaining inactivated streptococci. We may mention, for example, thermal inactivation, irradiation with ultraviolet or gamma rays, treatment with acids or treatment with hydrogen peroxide. The type of radiation, the intensity, the dose and the exposure time are adjusted by a person skilled in the art according to the quantity and the nature of the bacteria to be inactivated.

In particular, the inactivated streptococci according to the present description may be inactivated by thermal treatment.

In general, it is considered that a heat treatment at a temperature below about 50° C. might not inactivate the bacteria, or might inactivate only a fraction of the bacteria exposed to the treatment. In general, it is considered that a heat treatment at a temperature exceeding about 80° C. may induce degradation of some or all of the bacterial components, and in particular may induce denaturation of the bacterial surface compounds, making the inactivated bacteria unsuitable for use according to the present description.

A thermal treatment according to the present description may comprise exposure of the bacteria to be inactivated to a temperature varying from about 50° C. to about 80° C., in particular varying from about 60° C. to about 70° C., and in particular at a temperature of about 65° C. A thermal treatment according to the present description may result in exposure of the bacteria to a dry heat or a wet heat. Thus, thermal inactivation of the bacteria may be carried out by subjecting the bacteria to be inactivated to a suitable heat source, such as a water bath, a stove, or a furnace. Besides exposure to a heat source, the bacteria to be inactivated may also be subjected to a pressure above atmospheric pressure.

According to a particular embodiment, the bacteria to be inactivated are exposed to a source of thermal radiation by subjecting them to treatment on a water bath.

The methods of pasteurization used for agricultural and food products may be applied to the inactivation treatment by thermal treatment of S. salivarius (Chillet, 2011. Collection Biotech).

The exposure time of the bacteria to a method or treatment of inactivation is adapted by a person skilled in the art according to the factors usually adopted in this field for obtaining inactivation of all the bacteria undergoing the treatment, without substantially affecting the components responsible for the activity of the inactivated bacteria with respect to glucose intolerance or associated diseases.

The factors usually considered for determining a time of exposure to an inactivation treatment may be the nature of the bacteria to be inactivated, the quantity of bacteria, the degree of concentration of the bacteria, the nature of the medium containing the bacteria, the type of treatment, etc.

According to one embodiment, the streptococci are put in a buffer, in particular a phosphate buffer or a saline buffer, to be inactivated by thermal treatment. This buffer may be the same as that used in the purification step. Thus, the purified streptococci may be submitted directly to the inactivation step. Alternatively, the streptococci suspended in a purification buffer may be separated from the buffer and suspended in an inactivation buffer.

According to one embodiment, the concentration of the streptococci may be adjusted before the inactivation step. According to one embodiment, the streptococci may be used at a concentration in the range from about 1.10⁷ CFU/ml to 1.10¹² CFU/ml. A concentration in this range is particularly suitable for inactivation by thermal treatment.

In general, it is considered that a thermal treatment, for example at a temperature as defined above, for a time of less than about 15 minutes may not be sufficient to inactivate the bacteria, or may only lead to inactivation of a fraction of the bacteria exposed to the treatment. In general, it is considered that a thermal treatment for a time exceeding about 30 minutes may induce degradation of some or all of the bacterial components, and in particular may induce denaturation of the bacterial surface compounds, making the inactivated streptococci unsuitable for use according to the present description.

According to one embodiment, the streptococci may be inactivated by a thermal treatment applied, at a temperature as indicated above, for a time ranging from about 15 minutes to about 30 minutes, in particular from about 18 minutes to about 25 minutes, and in particular for about 20 minutes.

After the inactivation step, the inactivated bacteria may be used as they are, or if applicable, may be submitted to a purification step, for example as described above.

A treatment of inactivation of the streptococci, in particular a thermal inactivation treatment, may lead to inactivation of at least 99.9% of the live bacteria submitted to the inactivation treatment. Thus, starting from a concentration of bacteria of 10⁸ CFU, the survival rate is less than or equal to 10² CFU. In particular, an inactivation treatment may lead to inactivation of at least 99.99%, or even 100%, of the live bacteria submitted to the inactivation treatment. The inactivation of the bacteria may be monitored by various methods known by a person skilled in the art. One possible method of monitoring is reculturing of the bacteria, or of a representative sample of the bacteria, obtained at the end of the method of inactivation.

Preferably, reculturing may be carried out on agar so as to be able to count any colonies formed and quantify the number of bacteria still alive at the end of the method of inactivation.

Any growth of bacteria after reculturing, in agar or in broth, may be indicative of partial efficacy of the method of inactivation. In such a situation, if necessary, the parameters of the inactivation process will be adjusted by a person skilled in the art to increase its efficacy and obtain an acceptable rate of inactivation, for example as defined above.

The inactivated streptococci may be used whole or in the form of membrane fragments or of bacterial fractions comprising at least membrane fragments. The membrane fractions according to the present description may be any fraction or any membrane extract, optionally purified or partially purified, obtained from a culture of streptococci according to the description, inactivated beforehand, and whose method of preparation comprises at least one step of lysis of the bacteria obtained after inactivation. After the lysis step, the method may optionally comprise a purification step with the aim of separating the fraction containing the membranes of the bacteria from the total lysate. Said step may be carried out by centrifugation or by filtration. A membrane fraction according to the present description may comprise some or all of the surface compounds of the membrane of the bacteria.

The membrane fragments or the bacterial fractions comprising said fragments may be obtained in the form of bacterial lysates.

A bacterial lysate according to the present description may comprise, besides the membranes or membrane fragments, some or all of the fractionated elements and/or metabolites resulting from lysis of the bacterial strain. A bacterial lysate according to the present description may be obtained by any method usually employed in this field, such as, for example, enzymatic lysis, thermal shock, ultrasound, osmotic shock, or mechanical stress, such as centrifugation. The membrane fractions obtained from the inactivated bacteria may be used as they are, or if applicable, may be purified as described above.

According to a particular embodiment, the inactivated streptococci according to the present description may be used whole.

An inactivated streptococcus, or membrane fractions obtained therefrom, are active substances in the sense of the present description.

According to one embodiment, an inactivated streptococcus, or a membrane fraction obtained therefrom, may be used in an amount equivalent to at least about 10⁷ CFU (colony forming units) and to at most about 10¹² CFU, in particular at least about 10⁸ CFU and at most about 10¹¹ CFU, and in particular at least about 10⁹ CFU and at most about 10¹⁰ CFU. In particular, an inactivated streptococcus, or a membrane fraction obtained therefrom, may be in an amount equivalent to about 10⁹ CFU or about 10¹⁰ CFU.

As the streptococci used according to the description are inactivated or are in the form of membrane fractions, the amounts used are determined on the basis of the quantities of live bacteria determined before inactivation.

### Glucose Intolerance and Associated Diseases

As stated above, an inactivated streptococcus, or a membrane fraction obtained therefrom, are active substances that may be used in a therapeutic method for preventing and/or treating glucose intolerance and/or a disease associated with glucose intolerance in an individual in need thereof.

An individual or patient considered according to the present description may be a mammal. A mammal according to the present description may, for example, be selected from domestic animals (such as cattle, sheep, cats, dogs, and horses), in particular cats and dogs, primates, such as human and nonhuman primates, rabbits, and rodents, such as mice and rats. According to one embodiment, an individual or patient according to the present description may be a human being.

An inactivated streptococcus, or a membrane fraction obtained therefrom, may be suitable in particular for use in a therapeutic treatment for preventing and/or treating prediabetes, and/or a disease or a disorder associated with prediabetes, or a symptom of prediabetes.

Glucose intolerance or prediabetes may be asymptomatic. However, they may be associated with obesity, dyslipidemia with an elevated level of triglycerides and/or a low level of HDL cholesterol, and hypertension. Glucose intolerance and prediabetes are associated with an increased risk of cardiovascular diseases. Prediabetes and glucose intolerance are regarded as an early stage of type 2 diabetes.

Glucose intolerance or prediabetes may be diagnosed by various methods known by a person skilled in the art. In particular, glucose intolerance in humans is diagnosed on the basis of two criteria:
fasting glycemia, and
glycemia two hours after oral ingestion of glucose.

Thus, glucose intolerance may be diagnosed if: (i) fasting glycemia is: between 6.1 and 6.9 mmol/L (1.10 to 125 mg/dL) and if (ii) two hours after ingestion of oral glucose of 75 g, glycemia is from 7.8 mmol/L to 11.0 mmol/L (140 mg/dL to 199 mg/dL).

Measurement of glycemia two hours after ingestion of glucose is carried out by an oral glucose tolerance test or OGTT. Said test is described in Nagy et al., Study of In Vivo Glucose Metabolism in High-fat Diet-fed Mice Using Oral Glucose Tolerance Test (OGTT) and Insulin Tolerance Test (ITT). J. Vis. Exp. (131), e56672, doi:10.3791/56672 (2018).

Glucose intolerance or prediabetes may generally be followed by type 2 diabetes. Type 2 diabetes (or DT2) is a disease associated with glucose intolerance or with prediabetes.

According to one embodiment, an inactivated streptococcus, or a membrane fraction obtained therefrom, may be suitable in particular for use in a therapeutic treatment for preventing and/or treating type 2 diabetes.

DT2 may be diagnosed by various methods known by a person skilled in the art. In particular, DT2 may be diagnosed if one or more criteria are met among:
fasting glycemia,
glycemia two hours after ingestion of oral glucose,
random glycemia, or
assay of glycated hemoglobin (HbAlc).

Thus, DT2 is diagnosed if: (i) fasting glycemia is: greater than or equal to 7.0 mmol/L (126 mg/dL), and/or if (ii) two hours after ingestion of 75 g of oral glucose, glycemia is greater than or equal to 11.1 mmol/L (200 mg/dL), and/or if (iii) randomly, glycemia is above 11.1 mmol/L (200 mg/dL). Raised levels of glycated hemoglobin Alc (HbAlc) in the nondiabetic range (>48 mmol/mol (equivalent to 6.5%)) may also be used for identifying people at risk of developing DT2.

According to one embodiment, the present invention also relates to a method of therapeutic treatment for preventing and/or treating glucose intolerance and/or a disease associated with glucose intolerance in an individual in need thereof comprising at least one step of administering to said individual at least one inactivated streptococcus, or a membrane fraction obtained therefrom, said streptococcus being obtained from a streptococcus comprising at least one gene coding for a 16S ribosomal RNA, said gene comprising a nucleotide sequence consisting of the sequence SEQ ID NO: 1.

According to one embodiment, the present invention also relates to a method of therapeutic treatment for preventing and/or treating prediabetes and/or a disease associated with prediabetes in an individual in need thereof comprising at least one step of administering to said individual at least one inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1.

According to one embodiment, the present invention also relates to a method of therapeutic treatment for preventing and/or treating type 2 diabetes in an individual in need thereof comprising at least one step of administering to said individual at least one inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1.

A method as described here may comprise, prior to the step of administration of an active substance according to the present description, a step of diagnosis of glucose intolerance/prediabetes or type 2 diabetes. Said diagnosis may be carried out by any method of diagnosis known by a person skilled in the art for this purpose, and in particular by one of the methods described above.

A method as described here may further comprise a step of observation of the reduction, suppression, or improvement of a symptom or of a glycemic marker of glucose intolerance/prediabetes or of type 2 diabetes.

According to a particular embodiment, an individual or a patient considered for the invention may be obese.

A use or a method according to the present description may comprise the administration, to an individual or patient in need thereof, of inactivated streptococci, or of membrane fractions obtained from the latter, by any route of administration that may be suitable. We may mention, for example, administration by the oral, sublingual, nasal, or rectal route. In particular, administration of inactivated streptococcus, or of a membrane fraction obtained therefrom, may be done by the oral route.

According to one embodiment, an inactivated streptococcus, or a membrane fraction obtained therefrom, may be administered at least once daily, in particular two or three times daily. More particularly, an inactivated streptococcus, or a membrane fraction obtained therefrom, is administered in at least one daily dose.

Inactivated streptococci, or membrane fractions derived therefrom, may be taken during a meal, or away from mealtimes. Over a period of 24 hours, these active substances may be taken at any time. In particular, they may be taken in the morning, and in particular at the time of the morning meal.

According to a variant embodiment, administration of the active substances described here may be done away from mealtimes, in particular in a daily dose away from mealtimes.

The period of time during which the inactivated streptococci, or membrane fractions derived therefrom, may be administered depends on several factors, such as the patient's age, weight, sex, the presence of other pathologies, and the diet, and is adapted by a person skilled in the art according to the usual practice in this field.

A period of time suitable for administration of inactivated streptococci, or of membrane fractions derived therefrom, may be from at least one week to at least fifteen weeks. In particular, said period may range from at least two to at least twelve weeks, in particular from at least three to at least ten weeks, or even from at least four to at least nine weeks. A period of time suitable for administration of inactivated streptococci, or of membrane fractions derived therefrom, may more particularly be at least two weeks.

During a period of administration, the active substances as described may be administered consecutively on each day of the period. For example, a treatment for a period of at least three weeks may comprise at least one administration of the inactivated bacteria, or of membranes obtained therefrom, each day in this period, i.e. for 21 consecutive days.

Alternatively, administration may be fractionated over the whole period considered, for example in two or more periods of one or more, for example 2, 3, 4, 5, or 6, consecutive days, these periods with administration being followed by a period without administration, of one or more, for example 2, 3, 4, 5, or 6, consecutive days. In this regimen of administration, a period of administration followed by a period without administration defines a treatment cycle. A treatment cycle may be repeated several times, for example at least one to fourteen times, or 2, 3, 4, 5, 6, 7, 8, 9, 10, or 11 times. Thus, this treatment regimen defines one or more cycles of administration and of interruption. For example, treatment over a period of at least nine weeks may comprise at least one dose per day of inactivated bacteria, or of membrane fractions derived therefrom, over a period of five consecutive days followed by two consecutive days without doses, said cycle then being repeated eight times to cover the period of nine weeks. According to one embodiment of this variant, administration of the inactivated bacteria, or of membrane fractions derived therefrom, may be done over a period of a day, followed by a day without a dose, these two days forming a cycle that may be repeated at least 1 to 40 or 50 times, for example to cover a total treatment time of up to 14 to 15 weeks.

In a variant embodiment, the inactivated streptococci, or membrane fractions derived therefrom, may be administered for at least two consecutive days out of a period of a week. In said embodiment, the inactivated bacteria, or membrane fractions derived therefrom, are taken on two days, followed by 5 days without doses. The whole defines a cycle that extends over a week and may be repeated at least one to fourteen times. According to another embodiment, administration may be carried out for at least five consecutive days out of a period of a week. In said embodiment, the inactivated bacteria, or membrane fractions derived therefrom, are taken for 5 days, followed by 2 days without doses. The whole defines a cycle that extends over a week and may be repeated at least one to fourteen times. For example, in these embodiments, administration may be carried out for at least seven consecutive days out of a period of a week, i.e. each day of the week. This period of treatment may be repeated at least one to fourteen times. This embodiment does not have a period without doses.

In these various methods of treatment, administration may be continued for two, three or more weeks, as indicated above.

According to one embodiment, inactivated streptococci, or membrane fractions derived therefrom, may be administered in an effective amount or therapeutic dose. In particular, these active substances may be administered at a daily dose equivalent to a dose ranging from at least about 10⁷ CFU to about at least 10¹² CFU. In particular, a dose may be from at least about 10⁸ CFU to at least about 10¹¹ CFU, and in particular from at least about 10⁹ CFU to at least about 10¹⁰ CFU.

Compositions Comprising Inactivated Streptococci or Membrane Fractions The inactivated streptococci, or the membrane fractions derived therefrom, may be formulated in any composition comprising at least one pharmaceutically or physiologically acceptable vehicle. Said composition is in particular adapted to the methods of administration and the dosages indicated above.

“Pharmaceutically acceptable” or “physiologically acceptable” means that the vehicle (carrier, diluent, or excipient) must be compatible with the other ingredients of the formulation, and must not be harmful to the individual to whom the composition comprising it is administered. A pharmaceutically acceptable vehicle is a vehicle recognized as satisfying, in particular, the criteria of safety, compatibility, and inertness required for use in the pharmaceutical field. As examples of pharmaceutically acceptable vehicles, we may mention sterile water, saccharides such as sucrose or saccharose, starches, sugar alcohols such as sorbitol, polymers such as PVP or PEG, lubricants such as magnesium stearate, preservatives, colorants or flavors.

A composition according to the present description comprising inactivated streptococci, or membrane fractions derived therefrom, and at least one physiologically or pharmaceutically acceptable vehicle, comprises the streptococci or the membrane fractions in a therapeutically effective amount or dose. In particular, a composition may be adapted for administration of an equivalent of at least about 1.10⁷ CFU to about at least 1.10¹² colony forming units (CFU), in particular from at least about 1.10⁸ CFU to at least about 1.10¹¹ CFU, preferably from at least about 1.10⁹ CFU to at least about 1.10¹⁰ CFU, and more preferably at least about 1.10¹⁰ CFU of live streptococci.

A composition as described here is adapted to allow a daily dose of a therapeutically effective amount or dose of inactivated streptococci, or of membrane fractions obtained therefrom.

A composition as described here may be suitable for administration during a meal or away from mealtimes, in particular away from mealtimes. In certain embodiments, a composition may be suitable for administration in at least one daily dose, in particular once daily.

As detailed above, a composition as described here may be administered over a period of one or more consecutive days, if applicable followed by a period without doses, the whole defining a cycle that may or may not be repeated. According to certain embodiments, a composition may be administered on each consecutive day over the period of administration considered. Alternatively, a composition as described here may be administered over a period of one, two or more consecutive days followed by a period of interruption of at least one, two or more consecutive days. The periods of administration and of interruption may constitute a cycle. A composition may be administered in at least one, or even two, three or more consecutive cycles.

A composition as described here may be administered for a period of at least one to at least fifteen weeks, in particular for at least two to at least twelve weeks, in particular for at least three to at least ten weeks, or even for at least four to at least nine weeks.

A composition as described here may advantageously be administered according to the methods of administration as described above.

As a physiologically acceptable vehicle that may be used for formulating a composition according to the present description, we may mention, nonexhaustively, water, saline buffers, in particular a phosphate buffer, sodium bicarbonate, juices, milk products, in particular milk or yoghurts, food compositions for babies, thickeners, such as glycerol monostearate, sweeteners, coating agents, such as colza oil; soybean oil; peanut oil; soybean lecithin or fish gelatin, diluents, such as lactose; lactose monohydrate or starch, binders, such as povidone; gelatinized starch; gums; sucrose; polyethylene glycol (PEG) 4000 or PEG 6000, dispersants, such as microcrystalline cellulose or sodium carboxymethyl starch, such as sodium carboxymethyl starch type A, lubricants, such as magnesium stearate, fluidizing agents, such as anhydrous colloidal silica, etc.

A pharmaceutically acceptable vehicle may be any substance used for preparing galenical dosage forms, including coating materials, film-forming materials, fillers, disintegrants, materials for modifying release, carrier materials, diluents, binders and other additives. The pharmaceutically acceptable vehicles usually employed in this field comprise substances such as sucrose, mannitol, sorbitol, starch and starch derivatives, lactose, lubricants such as magnesium stearate, disintegrating agents, and buffering agents. Moreover, suitable pharmaceutically acceptable vehicles comprise, for example, water, saline solutions, alcohols, oils, preferably vegetable oils, polyethylene glycols, gelatin, lactose, amylose, magnesium stearate, surfactants, perfume oil, fatty acid, monoglycerides and diglycerides, hydroxymethylcellulose, polyvinylpyrrolidone and the like. The pharmaceutical or nutraceutical compositions may comprise auxiliaries, such as lubricants, preservatives, stabilizers, wetting agents, emulsifiers, salts for influencing osmotic pressure, buffers, colorants, aromatizing agents and/or aromatic substances.

Liquid forms are envisaged for the present invention. These may comprise emulsions, solutions, suspensions and syrups. Solid forms are also envisaged, such as suppositories, tablets, pastilles, pills, hard capsules, powders, effervescent formulations, sugar-coated pills, granules, or soft capsules.

A composition as described here may further comprise any additive or excipient usually employed in this field for the use considered for the composition. Thus, a composition may further comprise at least one selected from a sweetener, a stabilizer, an antioxidant, an additive, an aromatizing agent and/or a colorant. A composition as described here may be formulated by the usual methods employed in this field, in particular for producing sugar-coated tablets, hard capsules, gels, hydrogels for controlled release, emulsions, mousses, syrups, liquids, tablets, soft capsules, or suppositories.

A composition according to the present invention may be adapted to administration by the oral, sublingual, nasal, or rectal route, in particular by the oral route.

In one embodiment, a composition as described here may be in the form of a dietary supplement, a complement for a drink, a nutritional product, a medical food or a nutraceutical composition.

A composition according to the present invention may also be in the form of a nutritional or nutraceutical composition.

In particular, a composition as described here may be a dietary supplement.

A dietary supplement may in particular be in the form of hard capsules, soft capsules, tablets, sugar-coated tablets, pills, pastes, pastilles, gums, oral solutions or emulsions, sirup or gel.

A composition as described here may in particular be formulated in the form of a dietary supplement selected from milk products, milk drinks, yoghurts, fruit or vegetable juices or concentrates thereof, powders, drinks based on soybean or cereals, breakfast cereals such as muesli flakes, powdered fruit and vegetable juices, bars of cereals and/or chocolate, confectionery, spreads, milk powders, smoothies, ice creams, products based on reconstituted fruits, food bars, sauces, sport supplements, including dairy and nondairy sport supplements, a dessert, a frozen food, a soup, a liquid suspension, a tablet, a gum or candy.

Advantageously, a composition for the oral route may be provided with an enteric coating in order to ensure that the inactivated streptococci, or membrane fractions obtained therefrom, can pass through the stomach intact. Release of the active substances according to the invention may thus take place in the upper intestinal tract.

According to a particular embodiment, a composition for the oral route as described here may be selected from a powder, granules, a food product, a drink, a pharmaceutical product, a nutraceutical, a food additive, a dietary supplement, a soft capsule, and a hard capsule. In another embodiment, a composition may be administered by the rectal route. In particular, a composition for the rectal route may be prepared in the form of a suppository, an enema or a mousse.

In one embodiment, a composition as described here may further comprise at least one additional active substance selected from: metabolites, antioxidants, fish oils, DHA, EPA, vitamins, minerals, phytonutrients, a protein, a lipid, probiotics and combinations thereof.

As probiotic active substances that may be used additionally, we may mention the strains of probiotic bacteria derived from Streptococcus, Lactobacillus, Lactococcus, Bifidobacterium, Veillonella, Desemzia, Coprococcus, Collinsella, Citrobacter, Turicibacter, Sutterella, Subdoligranulum, Sporobacter, Sporacetigenium, Ruminococcus, Roseburia, Proteus, Propionobacterium, Leuconostoc, Weissella, Pediococcus, Prevotella, Parabacteroides, Papillibacter, Oscillospira, Melissococcus, Dorea, Dialister, Clostridium, Cedecea, Catenibacterium, Butyrivibrio, Buttiauxella, Bulleidia, Bilophila, Bacteroides, Anaerovorax, Anaerostopes, Anaerofilum, Enterobacteriaceae, Fermicutes, Atopobium, Alistipes, Acinetobacter, Slackie, Shigella, Shewanella, Serratia, Mahella, Lachnospira, Klebsiella, Idiomarina, Fusobacterium, Fecalibacterium, Eubacterium, Enterococcus, Enterobacter, or Eggerthella.

As phytonutrients, we may mention carotenoids, polyphenols or resveratrol. As antioxidants, we may mention vitamin C, glutathione, caffeine, or tocopherol. As vitamins, we may mention vitamins A, B, C, or D. As minerals, we may mention iron, magnesium, calcium, zinc, copper, or sodium.

According to a particular embodiment, a composition as described here advantageously is not a nutritional composition comprising a mixture of oligosaccharides comprising an N-acetylated oligosaccharide, a galacto-oligosaccharide and a sialylated oligosaccharide.

According to another embodiment, a composition as described here is not intended to be used for reducing and/or avoiding excessive accumulation of adipose mass in an infant or a young child, and/or for preventing a health disorder at a later age connected with excessive accumulation of adipose mass in an infant or a young child, such as obesity at a later age, and the associated comorbidities.

It is to be understood that the present description includes all the variants, combinations and permutations in which one or more limitations, elements, clauses, descriptive terms, etc., of one or more of the claims enumerated hereunder may be introduced into another claim dependent on the same basic claim (or any other claim, as appropriate), unless stated otherwise or unless it would be obvious to a person skilled in the art that a contradiction or an inconsistency would arise. When elements are presented in the form of lists (for example, in the Markush group or a similar format), it has to be understood that each subgroup of elements is also disclosed, and any element may be removed from the group. It has to be understood that in general, when the present description or aspects of the present description is/are denoted as comprising particular elements, features, etc., certain embodiments of the present description or of certain aspects of the present description consist, or consist essentially of said elements, features, etc. For simplicity, these embodiments have not always been stated specifically in as many words. It must also be understood that any embodiment or aspect of the present description may be excluded explicitly from the claims, regardless of the fact that the specific exclusion is mentioned in the specification. The publications and other documents cited for describing the background of the present description and for supplying additional details concerning its implementation are incorporated here by reference.

The present description is described in more detail hereunder by means of the following examples, which are presented solely for purposes of illustration, and do not limit the present description.

### Subject Matter of the Present Description

A first object of the present description relates to an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, for use in a therapeutic method for preventing and/or treating glucose intolerance and/or a disease associated with glucose intolerance in an individual in need thereof.

A second object of the present description relates to inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, for use in a therapeutic method for improving the response to glucose in an individual with glucose intolerance in an individual in need thereof.

A third object of the present description relates to an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, for use in a therapeutic method for preventing and/or treating prediabetes and/or a disease associated with prediabetes in an individual in need thereof.

A fourth object of the present description relates to an inactivated streptococcus, or a membrane fraction obtained therefrom, for use according to object 1, 2 or 3, in which said disease associated with glucose intolerance is type 2 diabetes.

A fifth object of the present description relates to an inactivated streptococcus, or a membrane fraction obtained therefrom, said streptococcus being obtained from a streptococcus comprising a gene coding for a 16S ribosomal RNA, said gene comprising a nucleotide sequence consisting of the sequence SEQ ID NO: 1, for use in a therapeutic method for preventing and/or treating type 2 diabetes in an individual in need thereof.

A sixth object of the present description relates to an inactivated streptococcus, or a membrane fraction obtained therefrom, for use according to object 1 to 5, in which said individual is a mammal, in particular a human being.

A seventh object of the present description relates to an inactivated streptococcus, or a membrane fraction obtained therefrom, for use according to the objects 1 to 6, in which said individual is obese.

An eighth object of the present description relates to an inactivated streptococcus, or a membrane fraction obtained therefrom, for use according to one of the objects 1 to 7, in which the streptococcus is inactivated by a treatment selected from: a thermal treatment, an irradiation with ultraviolet, an irradiation with gamma rays, an acid treatment, or a treatment with hydrogen peroxide, in particular by a thermal treatment.

A ninth object of the present description relates to an inactivated streptococcus, or a membrane fraction obtained therefrom, for use according to object 8, in which said thermal treatment comprises exposure to a temperature varying from about 50° C. to about 80° C., in particular varying from about 60° C. to about 70° C., and preferably at a temperature of about 65° C.

A tenth object of the present description relates to an inactivated streptococcus, or a membrane fraction obtained therefrom, for use according to object 8 or 9, in which said thermal treatment is of a duration varying from about 15 minutes to about 30 minutes, in particular from about 18 minutes to about 25 minutes, and in particular is of about 20 minutes.

An eleventh object of the present description relates to an inactivated streptococcus, or a membrane fraction obtained therefrom, for use according to one of the objects 1 to 10, in which the inactivated streptococcus or said membrane fraction is administered by the oral route.

A twelfth object of the present description relates to an inactivated streptococcus, or a membrane fraction obtained therefrom, for use according to one of the objects 1 to 11, in which the inactivated streptococcus or said membrane fraction is administered in at least one daily dose, and in particular once daily.

A thirteenth object of the present description relates to an inactivated streptococcus, or a membrane fraction obtained therefrom, for use according to one of the objects 1 to 12, in which the inactivated streptococcus or said membrane fraction is administered for at least two consecutive days out of an interval of a week, and in particular for at least five consecutive days out of an interval of a week.

A fourteenth object of the present description relates to an inactivated streptococcus, or a membrane fraction obtained therefrom, for use according to one of the objects 1 to 13, in which the inactivated streptococcus or said membrane fraction is administered for at least one to at least fifteen weeks, in particular for at least two to at least twelve weeks, in particular for at least three to at least ten weeks, or even at least four to at least nine weeks and more particularly for at least two weeks.

A fifteenth object of the present description relates to an inactivated streptococcus, or a membrane fraction obtained therefrom, for use according to one of the objects 1 to 14, in which the inactivated streptococcus or said membrane fraction is administered at a daily dose equivalent to a dose from at least about 1.10⁷ CFU to about at least 1.10¹² CFU, in particular from at least about 1.10⁸ CFU to at least about 1.10¹¹ CFU, and in particular from at least about 1.10⁹ CFU to at least about 1.10¹⁰ CFU.

A sixteenth object of the present description relates to an inactivated streptococcus, or a membrane fraction obtained therefrom, for use according to one of the objects 1 to 15, in which the inactivated streptococcus or said membrane fraction is in a composition comprising at least one physiologically or pharmaceutically acceptable vehicle.

A seventeenth object of the present description relates to an inactivated streptococcus, or a membrane fraction obtained therefrom, for use according to object 16, in which said composition is selected from a powder, granules, a food product, a drink, a pharmaceutical product, a nutraceutical, a food additive, a dietary supplement, a soft capsule, and a hard capsule.

An eighteenth object of the present description relates to an inactivated streptococcus, or a membrane fraction obtained therefrom, for use according to object 16 or 17, in which said composition further comprises at least one additional active substance selected from: metabolites, antioxidants, fish oils, DHA, EPA, vitamins, minerals, phytonutrients, a protein, a lipid, probiotics, and combinations thereof.

A nineteenth object of the present description relates to a composition comprising an inactivated streptococcus, or a membrane fraction obtained therefrom, and at least one physiologically or pharmaceutically acceptable vehicle for use in a therapeutic method for preventing and/or treating glucose intolerance and/or a disease associated with glucose intolerance in an individual in need thereof.

### EXAMPLES

The following examples illustrate the property of inactivated streptococci obtained from streptococci comprising a gene coding for a 16S ribosomal RNA, said gene comprising a nucleotide sequence consisting of the sequence SEQ ID NO: 1, in particular the species S. salivarius, more particularly the strains JIM8772 and JIM8777, to regulate glycemia in obese rat and mice models.

### Example 1: Heat-Inactivated S. salivarius Improves Glucose Intolerance in a Rat Model

### Materials and Methods

### Preparation of the Bacteria

### Culture

A preculture of S. salivarius JIM8772 was prepared from a bacterial colony isolated on M17 glucose agar medium, and then seeded in 5 ml of M17 liquid glucose medium, and cultured overnight at 37° C.

Starting from this preculture, a bacterial culture in M17 glucose medium (for 1 L: 5 g Tryptone, 5 g soybean peptone, 5 g meat infusion, 2.5 g of yeast extract, 0.5 g of ascorbic acid, 0.25 g of magnesium sulfate, 19 g disodium glycerophosphate and 10 g glucose) was seeded and was cultured at 37° C., until OD (optical density) at 600 nm was between 1 and 2.

The bacteria were collected and centrifuged for 20 min at 6000 rpm. The supernatant was removed and the pellets were washed with an equivalent volume of 0.9% NaCl buffer. After a second centrifugation for 20 min at 6000 rpm and removal of the 0.9% NaCl buffer, the bacterial pellet was taken up in a 10 times smaller volume of 0.9% NaCl buffer than the previous volume to concentrate the bacteria. After a third centrifugation for 20 min at 6000 rpm, the bacterial pellet was taken up in a 10 times smaller volume of 0.9% NaCl buffer than the previous volume to concentrate the bacteria.

The bacteria were then distributed in 2-ml tubes at a rate of 1 ml, and then inactivated by heat, or stored live.

### Inactivation by Heat

The bacteria distributed in tubes were exposed for 20 min to 65° C. on a water bath. The heat-inactivated S. salivarius bacteria are designated “HK” hereinafter (heat killed—thermally inactivated). Inactivation of the bacteria was tested by reculturing a sample of bacteria on M17 agar and by observing absence of growth after 24 h at 37° C. The integrity of the bacteria, and in particular the absence of bacterial lysis was verified by scanning electron microscopy. The bacteria were collected and centrifuged for 20 min at 6000 rpm from bacterial cultures in M17 glucose medium. The supernatant was removed and the bacterial suspensions, immersed in a fixative solution (2.5% of glutaraldehyde in 0.2 M sodium cacodylate buffer, pH 7.4), were deposited on sterile glass disks (Marienfeld, VWR, France) and stored at room temperature for 1 hour and at 4° C. overnight. The fixative was removed and the samples were rinsed three times for 10 min in a solution of sodium cacodylate (pH 7.4). The samples underwent gradual dehydration by immersion in a graduated series of ethanol (50 to 100%) before drying at the critical point under CO₂. The samples were mounted on glass carrier disks (50 mm diameter) fixed with carbon adhesive disks (Agar Scientific, Oxford Instruments SAS, GOMETZ-LA-VILLE, France) and metallized with platinum (Polaron SC7640, Milexia, Verrieres-le-Buisson, France) for 220 seconds at 10 mA. The samples were imaged by field-emission scanning electron microscopy (SEM FEG). They were imaged by detection of secondary electrons and backscattered electrons (5 kV) with a Hitachi SU5000 microscope (Milexia, Verrieres-le-Buisson, France). Sample preparation and analyses by scanning electron microscopy were carried out within the collective scientific infrastructure (infrastructure scientifique collective, ISC) of microscopy and imaging MIMA2 (INRA, Jouy-en-Josas, France—DOI: MIMA2, INRAE, 2018. Microscopy and Imaging Facility for Microbes, Animals and Foods, https://doi.org/10.15454/1.5572348210007727E12).

### Live Bacteria

Reculturing of a sample of bacteria on M17 agar makes it possible, after 24 h of growth at 37° C., to calculate the concentration of the live Streptococcus salivarius JIM8772 bacteria. The tubes comprising the live S. salivarius bacteria are kept in ice until used within 3 hours following preparation. The live bacteria are indicated with the letter L (for “living”): S. salivarius L.

### Animals

Male CdSD rats (275-300 g) from Charles River were used.

### Diet

The animals received either a control diet or an HFD diet (high-fat obesity-inducing diet—High Fat Diet). The HFD diet is made up of: 45% of lipids+35% of carbohydrates+20% of proteins with 4.7 kcal/g.

The control diet is made up of 11% of lipids+56% of carbohydrates+33% of proteins with 3.3 kcal/g.

### Study Design

After 13 days of acclimation, a group of 21 rats was kept on an HFD diet for 9 weeks and a group of 7 rats received the control diet. The rats in the HFD group were then distributed as a function of their weight to create 3 homogeneous groups each of 7 animals.

After these 9 weeks and for 3 weeks, an HFD group (HFD control) and the control group (Standard) were fed by stomach tube each day with 1 ml of a saline solution (0.9% NaCl), an HFD group was fed by stomach tube each day with a saline solution of 1 ml with 1.10¹⁰ CFU of live bacteria (S. salivarius L), and an HFD group was fed by stomach tube each day with a saline solution of 1 ml of inactivated bacteria (S. salivarius HK) comprising an equivalent of 1.10¹⁰ CFU.

The study comprised the following 4 groups:
Group Standard+saline solution (Standard)
Group HFD+saline solution (HFD Control)
Group HFD+saline solution containing S. salivarius JIM8772 HK (HFD HK)
Group HFD+saline solution containing S. salivarius JIM8772 L (HFD L)

### Measurement of Adipose Mass

After the 12 weeks (9+3) of diet, the rats were killed and the epididymal, visceral and retroperitoneal adipose tissues were taken and weighed in order to determine the effects of the diet, if applicable supplemented with live or heat-inactivated S. salivarius bacteria, on the gain and accumulation of adipose tissues.

The weight of the adipose tissues was measured: after specific dissection of each tissue, they were weighed on an electronic balance.

### Measurement of Glycemia

After 11 weeks of diet (i.e. one week before slaughter, and after two weeks of diet supplemented or not with bacteria), an oral glucose tolerance test (OGTT) was carried out on 6 of the 7 rats of each group in order to evaluate the animals' capacity to regulate their glycemia.

This test consists of monitoring the plasma glucose concentrations for 120 minutes after oral ingestion of glucose. Thus, at T₀ (fasting glycemia), glucose (2 g/kg) is given to the animal by the oral route. A drop of blood is taken from the tail every 30 minutes for 120 minutes in order to measure glycemia with a strip glucometer (Accu-Chek Performa, available from pharmacies) (Nagy et al., J. Vis. Exp., 2018). The glucose tolerance reflects the capacity of the organism to restore normal glycemia.

### Statistical Analysis

Statistical analysis was carried out using the GraphPad Prism software (v7; San Diego, CA). All the data were analyzed using ANOVA followed by a Tukey multiple comparison test. Differences with values of p<0.05 were regarded as statistically significant.

### Results

### Maintenance of Integrity of the Heat-Inactivated Bacteria

The HK and live bacteria were observed in electron microscopy in order to compare their morphology and evaluate maintenance of the integrity of the bacteria after the thermal inactivation treatment.

It was observed that the envelope of the bacteria was identical between the inactivated strains and the live strains. More specifically, it was observed that the inactivated strains remained intact and did not display morphological alteration (FIG. 1).

Effect of the HFD Diet and Administration of Inactivated S. salivarius on the Rat's Weight and Adiposity

Firstly, it was observed that the weight of the adipose tissues and in particular of the epididymal, visceral and retroperitoneal adipose tissues was at least twice higher in the 3 HFD groups compared to the Standard group. In particular, the total weight of the 3 adipose tissues is on average 25 g for the Standard group and on average from 51 to 55 g for the 3 HFD groups (FIG. 2).

Thus, an increase in weight of the adipose tissues of the rats on the HFD diet was observed. The higher weight gain and the significant increase in the weight of the adipose tissues of the animals on the HFD diet are signs of development of obesity.

Improvement of Glucose Intolerance by S. salivarius HK

After 11 weeks of diet, an oral glucose tolerance test (OGTT) was undertaken. A smaller increase in blood glucose concentration was observed, during the 90 minutes following the application of oral glucose, in the rats in the HFD group+S. salivarius HK (137±4.7 mg/dl at 90 min post gavage) compared to the rats in the HFD Control group (167±6.7 mg/dl at 90 min post gavage). The variation in the blood glucose concentration of the HFD group+S. salivarius HK in the 90 min following oral administration of glucose was comparable to that of the Standard group (133±3.2 mg/dl at 90 min post gavage) (FIG. 3A).

Moreover, the area under curve (AUC) of the HFD group+S. salivarius HK (249.4.±4.5 mg/dl/h) was decreased compared to the HFD Control group (303.1±9.0 mg/dl/h) and the HFD group+S. salivarius L (286.1±9.7 mg/dl/h) and comparable to that of the Standard group (250.5±6.8 mg/dl/h) (FIG. 3B). These results indicate that glucose intolerance that develops in the groups HFD Control and HFD+S. salivarius L is significantly reduced in the group of rats HFD+S. salivarius HK. The treatment with live bacteria did not significantly alter the glucose intolerance of the rats on the HFD diet.

Thus, whereas gavage of the rats HFD+S. salivarius L does not change glucose intolerance relative to the HFD Control rats, unexpectedly the group of rats HFD+S. salivarius HK has glycemia similar to that of the Standard group (FIG. 3B). This indicates a therapeutic effect of inactivated S. salivarius on the establishment of glucose intolerance.

Thus, the data presented demonstrate that an inactivated strain of the species Streptococcus salivarius makes it possible to improve glucose intolerance substantially. Inactivated S. salivarius thus proves to be particularly interesting for preventing and/or treating glucose intolerance and/or a disease associated with glucose intolerance, in particular in an obese and/or prediabetic individual.

### Example 2: Heat-Inactivated S. salivarius Improves Glucose Intolerance in a Mouse Model

### Materials and Methods

### Preparation of the Bacteria

### Culture

A preculture of S. salivarius JIM8777 was prepared from a bacterial colony isolated on M17 glucose agar medium and seeded in 5 ml of M17 liquid glucose for culture at 37° C. overnight. Starting from this preculture, a bacterial culture in M17 glucose medium (for 1 L: 5 g Tryptone, 5 g soybean peptone, 5 g meat infusion, 2.5 g of yeast extract, 0.5 g of ascorbic acid, 0.25 g of magnesium sulfate, 19 g disodium glycerophosphate and 10 g glucose) was seeded and was cultured at 37° C., up to an OD at 600 nm between 4 and 6.

The bacteria were collected and centrifuged for 10 min at 6000 rpm. The supernatant was removed and the pellets were washed with an equivalent volume of PBS buffer. After a second centrifugation for 10 min at 6000 rpm, the bacterial pellet was taken up in a 10 times smaller volume of PBS buffer than the initial culture volume, to concentrate the bacteria.

The bacteria were then distributed in 2-ml tubes at a rate of 1 ml, and then either inactivated by heat or stored live.

### Inactivation by Heat

The tubes containing 1 ml of bacteria are treated for 20 min at 65° C. on a water bath. The bacteria are centrifuged for 10 min at 6000 rpm, the PBS buffer is removed and the dry pellets are frozen directly in liquid nitrogen and stored at −80° C. until used. Inactivation of the bacteria was tested by reculturing a sample of bacteria on M17 agar and by observing absence of growth after 24 h at 37° C.

The heat-inactivated S. salivarius JIM8777 bacteria are designated “HK” hereinafter (heat killed—thermally inactivated).

### Live Bacteria

The live Streptococcus salivarius JIM8777 bacteria were centrifuged for 10 min at 6000 rpm, the PBS buffer was removed and the dry pellets were frozen directly in liquid nitrogen and stored at −80° C. until used. Reculturing of a sample of live bacteria on M17 agar makes it possible, after 24 h of growth at 37° C., to calculate the concentration of the live Streptococcus salivarius JIM8777 bacteria. The live bacteria are indicated hereunder with the letter L (for “living”): S. salivarius L.

### Animals

Male mice C57BL/6JRj aged 7 weeks from Janvier Labs were used.

### Diet

The animals received either a control diet or an HFD diet (obesity-inducing high-fat diet−High Fat Diet). The HFD diet is made up of: 45% of lipids+35% of carbohydrates+20% of proteins with 4.7 kcal/g.

The control diet is made up of 11% of lipids+56% of carbohydrates+33% of proteins with 3.3 kcal/g.

### Study Design

After 10 days of acclimation, 27 mice were kept on an HFD diet for 12 weeks, and 9 mice received a control diet (Control). The mice in the HFD group were distributed as a function of their weight to create 3 homogeneous groups each of 9 animals.

An HFD group (HFD Control) and the control group were fed by stomach tube for 5 days/week over a period of 12 weeks with 0.2 ml of a saline solution of PBS (80 g/l NaCl, 2 g/l KCl, 35.8 g/l Na₂HPO₄, 12H₂O and 2.4 g/l KH₂PO₄), an HFD group was fed by stomach tube every 5 days/week for 12 weeks with a saline solution of 0.2 ml with 1.10⁹ CFU of live S. salivarius bacteria (S. salivarius JIM8777 L), and an HFD group was fed by stomach tube on 5 days/week for 12 weeks with a saline solution of 0.2 ml of inactivated bacteria (S. salivarius JIM8777 HK) comprising an equivalent of 1.10⁹ CFU.

The study comprised the following 4 groups:
Control Group+saline solution (Control)
Group HFD+saline solution (HFD Control)
Group HFD+S. salivarius JIM8777 HK
Group HFD+S. salivarius JIM8777 L

### Measurement of Adipose Mass

After 12 weeks of diet, the body weight of the mice was measured by weighing in the morning on a precision electronic balance.

Afterwards, the mice were killed and the epididymal adipose tissue was taken and weighed on an electronic balance.

### Measurement of Glycemia

At 5 and 9 weeks of diet supplemented or not with inactivated or live bacteria, an oral glucose tolerance test (OGTT) was carried out on each group in order to evaluate the animals' capacity to regulate their glycemia.

This test consists of monitoring the plasma glucose concentrations for 120 minutes after oral ingestion of glucose. Thus, at T₀ (fasting glycemia), glucose (2 g/kg) is given to the animal by the oral route. A drop of blood is taken from the tail every 30 minutes for 120 minutes in order to measure glycemia with a strip glucometer (Accu-Chek Performa, available from pharmacies) (Nagy et al., J. Vis. Exp., 2018). Glucose tolerance reflects the body's capacity to restore normal glycemia.

### Statistical Analysis

Statistical analysis was carried out using the GraphPad Prism software (v7; San Diego, CA). All the data were analyzed using the Kruskal-Wallis test followed by a Dunn multiple comparison test. Differences with values of p<0.05 were regarded as statistically significant.

### Results

Effect of the HFD Diet and Administration of Inactivated S. salivarius on the Weight and Adiposity of the Mice

### Weight of the Mice

Firstly, the average final weight of the mice on the HFD Control diet (32.2±0.9 g) was greater than that of the mice in the Control group (27.8±0.4 g) (FIG. 4A). Administration of inactivated or live S. salivarius bacteria did not significantly affect the average final weight of the mice in the HFD groups (32.2±1.1 g for HFD+inactivated S. salivarius and 29.8 0.9 g for live S. salivarius) (FIG. 4A).

### Adiposity of the Mice

Moreover, it was also observed that the final average weight of the epididymal adipose tissue of the mice in the HFD Control groups (1.49±0.11 g) was significantly higher than that of the mice in the Control group (0.41 g±0.07 g).

Administration of inactivated or live S. salivarius bacteria had no substantial effect on the average final weight of the epididymal adipose tissue of the mice in the HFD groups (1.69±0.18 g for HFD+inactivated S. salivarius and 1.29±0.11 g for live S. salivarius) (FIG. 4B).

The greater weight gain and the significant increase in the weight of the adipose tissue of the mice on the HFD diet are signs of development of obesity in the mice.

Improvement of Glucose Intolerance by S. salivarius HK

After 5 and 9 weeks of treatment (S5 and S9), an oral glucose tolerance test was undertaken. At S5, it was observed that glucose intolerance in the mice receiving the HFD Control diet is significantly established compared to the Control mice. Moreover, it was observed that neither administration of the live S. salivarius bacteria, nor administration of the S. salivarius HK bacteria had an effect on this intolerance (FIGS. 5A and 5B).

However, at S9, a smaller increase in blood glucose concentration was observed, at 15, 30 and 60 minutes following application of oral glucose, in the mice in the group HFD+S. salivarius HK (405.1±8.7 mg/dl, 300.9±10.1 mg/dl and 244.8±7.4 mg/dl respectively at 15, 30 and 60 minutes post gavage compared to the mice in the Control group (365.3±8.1 mg/dl, 264.7±12.0 mg/dl and 196.0±7.6 mg/dl respectively at 15, 30 and 60 minutes post gavage) and compared to the mice in the HFD Control group (420.6±9.3 mg/dl, 385.0±20.3 mg/dl and 257.1±13.8 mg/dl respectively at 15, 30 and 60 minutes post gavage) (FIG. 6A).

Moreover, the area under curve (AUC) of the group HFD+S. salivarius HK (520.5 10.1 mg/dl/h) had decreased compared to the HFD Control group (577.5±18.3 mg/dl/h), comparable to the group HFD+live S. salivarius (529.6±18.1 mg/dl/h) and approaching that of the Control group (442.4±11.6 mg/dl/h) (FIG. 6B).

These results indicate that glucose intolerance is significantly reduced for the group of mice HFD+S. salivarius HK. Treatment with the live bacteria did not significantly alter the glucose intolerance of the mice submitted to the HFD diet.

Thus, whereas gavage of the HFD mice with S. salivarius L does not change glucose tolerance relative to the HFD Control mice receiving a saline gavage, unexpectedly the group of mice HFD that received the inactivated bacteria (S. salivarius HK) has improved glycemia relative to that of the HFD Control group (FIG. 6B). This indicates a therapeutic effect of inactivated S. salivarius on the establishment of glucose intolerance.

Thus, the data presented demonstrate that an inactivated strain of the species Streptococcus salivarius makes it possible to improve glucose intolerance substantially. Inactivated S. salivarius thus proves particularly interesting for preventing and/or treating glucose intolerance and/or a disease associated with glucose intolerance, in particular in an obese and/or prediabetic individual.

In conclusion, a protective effect was observed on glucose homeostasis of inactivated bacteria of the streptococci genus, as detailed in the description, in particular S. salivarius or S. vestibularis, and in particular S. salivarius. These inactivated bacteria limit glucose intolerance associated with obesity-inducing diets. In particular, it was observed that treatment with inactivated bacteria of the genus streptococcus, obtained from streptococci comprising a gene coding for a 16S ribosomal RNA, said gene comprising a nucleotide sequence consisting of the sequence SEQ ID NO: 1, makes it possible to regulate glucose metabolism with, in particular, an improvement of glucose intolerance.

### LIST OF DOCUMENTS CITED

Aas et al. (2005). Defining the normal bacterial flora of the oral cavity. Journal of Clinical Microbiology, 43(11), 5721-32.
Atlas du diabéte de la FID—8éme Édition, 2017.
Brookshier et al., P-0932, New Name For «Prediabetes» prompts quicker actions, IDF Congress, 2019
Carlsson et al. (1970). Early establishment of Streptococcus salivarius in the mouth of infants. Journal of Dental Research, 49(2), 415-8. R.
Chen and Burne, 1995. Analysis of Streptococcus salivarius urease expression using continuous chemostat culture. FEMS Microbiol. Letter, 135: 223-229.
Chillet, 2011. Biologie technique: Tome 2, La pasteurisation, Operations unitaires en génie biologique, Collection Biotech.
Delorme et al. 2007, Extent of Horizontal Gene Transfer in Evolution of Streptococci of the Salivarius Group. Journal of Bacteriology, February 2007, p. 1330-1341. doi: 10.1128/JB.01058-06. PubMed PMID: 17085557; PubMed Central PMCID: PMC1797340.
Giovannoni, SJ, TB Britschgi, CL Moyer et KG Field. 1990. Genetic diversity in Sargasso Sea bacterioplankton. Nature. 345:60-63.
Guédon et al. Complete genome sequence of the commensal Streptococcus salivarius strain JIM8777. J Bacteriol. 2011; 193(18):5024-5025. doi:10.1128/JB.05390-11.
Hamilton, I. R. 1968. Synthesis and degradation of intracellular polyglucosein Streptococcus salivarius. Can. J Microbiol. 14:65-77.
Harty et al. 1988. Fermentation products, amino acid utilization, maintenance energies and growth yields for the fibrillar Streptococcus salivavius HB and a non-fibrillar mutant HB-B grown incontinuous culture under glucose limitation. J. App. Microbiol. 65:143-152.
Kaci et al. 2011. Inhibition of the NF-kappaB Pathway in Human Intestinal Epithelial Cells by Commensal Streptococcus salivarius. AEM, 77: 4681-4684
Kozich, J. J., Westcott, S. L., Baxter, N. T., Highlander, S. K., and Schloss, P. D. (2013) Development of a dual-index sequencing strategy and curation pipeline for analyzing amplicon sequence data on the MiSeq Illumina sequencing platform. Appl. Environ. Microbiol. 79,5112-5120
McGovern et al. Comparison of medication adherence and persistence in type 2 diabetes: A systematic review and meta-analysis. Diabetes Obesity and Metabolism, 2017, 20(4):1040-1043
Moin T, Li J, Duru O K, et al. Metformin prescription for insured adults with prediabetes from 2010 to 2012: a retrospective cohort study. Ann Intern Med. 2015; 162(8):542-548. doi:10.7326/M14-1773
Nagy et al. Study of In Vivo Glucose Metabolism in High-fat Diet-fed Mice Using Oral Glucose Tolerance Test (OGTT) and Insulin Tolerance Test (ITT). J. Vis. Exp. (131), e56672, doi:10.3791/56672 (2018).
Plovier et al. A purified membrane protein from Akkermansia muciniphila or the pasteurized bacterium improves metabolism in obese and diabetic mice. Nature medicine, v. 23; n. 1; p.107-116; January 2017.
Porter et al. Hyperglycemia and Metformin Use Are Associated With B Vitamin Deficiency and Cognitive Dysfunction in Older Adults. J. Clin. Endocrinol. Metab., 2019, 104(10): 4837-4847.
Rapport mondial sur le diabéte, Organisation mondiale de la Santé 2016, ISBN: 9789242565256.
Roger et al. 2011. Characterization of Streptococcus salivarius growth and maintenance in artificial saliva. J. App. Microbiol. 111:631-641.
Smith et al. 1993. Oral streptococcal colonization of infants. Oral Microbiol. Immunol. 8:1-4
Srinivasan et al. (2012) Using PCR-Based Detection and Genotyping to Trace Streptococcus salivarius Meningitis Outbreak Strain to Oral Flora of Radiology Physician Assistant. PLoSONE 7(2): e32169.
Todd-Hewitt broth—Wescombe et al. 2011. Salivaricin 9, a new lantibiotic produced by Streptococcus salivarius. Microbiology, 157: 1290-1299
Wilson et al. 2012, Clinical and laboratory features of Streptococcus salivarius meningitis: a case report and literature review. Clinical Medicine & Research, 10(1), 15-25.
World Health Organization (1999). Definition, diagnosis and classification of diabetes mellitus and its complications: report of a WHO consultation. Part 1, Diagnosis and classification of diabetes mellitus.

### TRANSLATIONS OF REFERENCES IN FRENCH

Diabetes Atlas of the IDF—8th Edition, 2017.
Chillet, 2011. Industrial biology: Vol. 2, Pasteurization, unit operations in biological engineering, Collection Biotech.
World diabetes report, World Health Organization 2016, ISBN: 9789242565256.

1-13. (canceled)

14. A therapeutic method for preventing and/or treating glucose intolerance and/or a disease associated with glucose intolerance in an individual in need thereof, said method comprising a step of administering to the individual an inactivated streptococcus, or membrane fraction obtained therefrom, said streptococcus being obtained from a streptococcus comprising a gene coding for a 16S ribosomal RNA, said gene comprising a nucleotide sequence consisting of the sequence SEQ ID NO: 1.

15. The method of claim 14, wherein the disease associated with glucose intolerance is type 2 diabetes.

16. The method of claim 14, wherein the individual is a mammal.

17. The method of claim 14, wherein the streptococcus is inactivated by a treatment selected from: thermal treatment, irradiation with ultraviolet, irradiation with gamma rays, acid treatment, or treatment with hydrogen peroxide.

18. The method of claim 17, wherein the thermal treatment comprises exposure of the streptococcus to a temperature varying from about 50° C. to about 80° C.

19. The method of claim 14, wherein the streptococcus or said membrane fraction is administered by the oral route.

20. The method of claim 14, wherein the streptococcus, or said membrane fraction, is administered in at least one daily dose.

21. The method of claim 14, wherein the streptococcus, or said membrane fraction, is administered at a daily dose equivalent to a dose from at least about 10⁷ CFU to about at least 10¹² CFU.

22. The method of claim 14, wherein the streptococcus, or said membrane fraction, is in a composition comprising at least one physiologically or pharmaceutically acceptable vehicle.

23. A therapeutic method for preventing and/or treating glucose intolerance and/or a disease associated with glucose intolerance in an individual in need thereof, the method comprising a step of administering to the individual a composition comprising at least one inactivated streptococcus, or a membrane fraction obtained therefrom, said streptococcus being obtained from a streptococcus comprising a gene coding for a 16S ribosomal RNA, said gene comprising a sequence consisting of the sequence SEQ ID NO: 1, and at least one physiologically or pharmaceutically acceptable vehicle.

24. The method of claim 23, wherein the composition is a composition for the oral route.

25. The method of claim 23, wherein the composition is a composition suitable for at least one daily dose.

26. The method of claim 23, wherein the composition is a composition for administering a daily dose equivalent to a dose from at least about 10⁷ CFU to at least about 10¹² CFU.

27. The therapeutic method of claim 16, wherein the individual is an obese individual.

28. The therapeutic method of claim 17, wherein the streptococcus is inactivated by a thermal treatment.

29. The therapeutic method of claim 18, wherein the temperature varies from about 60° C. to about 70° C., or is of about 65° C.

30. The therapeutic method of claim 20, wherein the streptococcus, or said membrane fraction, is administered for at least two consecutive days out of a period of a week.

31. The therapeutic method of claim 20, wherein the streptococcus, or said membrane fraction, is administered at least once daily and is administered for at least two consecutive days out of a period of a week or is administered for at least five consecutive days out of a period of a week.

32. The therapeutic method of claim 21, wherein the streptococcus, or said membrane fraction, is administered at a daily dose equivalent to a dose from at least about 10⁸ CFU to at least about 10¹¹ CFU or from at least about 10⁹ CFU to at least about 10¹⁰ CFU.

33. The therapeutic method of claim 24, wherein the composition is suitable for administration for at least two consecutive days out of a period of at least one week.


SEQUENCE LISTING (7 sequences)

SEQ ID NO: 1 - DNA, 590 bp, Streptococcus sp.
  Fragment of the gene coding for 16S rRNA (1)..(590)
ctacaagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgc

SEQ ID NO: 2 - DNA, 1442 bp, Streptococcus salivarius
  Gene coding for 16S rRNA S. salivarius (1)..(1442)
tcctggctcaggacgaacgctggcggcgtgcctaatacatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttt

SEQ ID NO: 3 - DNA, 1442 bp, Streptococcus vestibularis
  Gene coding for 16S rRNA S. vestibularis (1)..(1442)
tcctggctcaggacgaacgctggcggcgtgcctaatacatgcaagtagaacgctgaagagaggagcttgctcttcttgaatgagttgcgaacgggtgagtaacgcgtaggtaacctgccttgtagcgggggataactattggaaacgatagctaataccgcataacaatgggtgacacatgtcatttatttgaaaggggcaattgctccactacaagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttt

SEQ ID NO: 4 - DNA, 15 bp, Artificial
  Synthetic Sense primer
tacggraggcagcag

SEQ ID NO: 5 - DNA, 19 bp, Artificial
  Synthetic Antisense primer
taccagggtatctaatcct

SEQ ID NO: 6 - DNA, 21 bp, Artificial
  Synthetic Sense primer
gctaataccgcataacaatgg

SEQ ID NO: 7 - DNA, 20 bp, Artificial
  Synthetic Antisense primer
cggaaaggatccaacaccta